(19) United States
(12) Patent Application Publication
(10) Pub. No.: US 2026/0215780 A1
(43) Pub. Date: Jul. 30, 2026

(54) NERVE CONDUIT SYSTEM

(71) Applicant: Tissium S.A., Paris (FR)

(72) Inventors: Marie Malafosse, Paris (FR); Elise Devries, San Francisco, CA (US); Estelle Collin, Alfortville (FR)

(73) Assignee: Tissium S.A., Paris (FR)

(21) Appl. No.: 19/143,262

(22) PCT Filed: Dec. 21, 2023
(86) PCT No.: PCT/EP2023/087467

§ 371 Date: Jun. 25, 2025

(30) Foreign Application Priority Data
Dec. 27, 2022 (WO) PCT/EP2022/087899

(51) Int. Cl. A61B 17/11; A61B 17/00

(52) U.S. Cl. CPC A61B 17/1128; A61B 2017/00477; A61B 2017/1132

(57) ABSTRACT

A nerve conduit systems supporting a repair of nerve lesions by facilitating proper placement and orientation of the nerve ends and enabling the connection of nerve ends having same or different diameters. Accordingly, a nerve conduit system for connecting two nerve ends in an end-to-end manner includes a first part and a second part, each part including an insertion portion each being adapted to receive one of the two nerve ends, and a connection portion, each part includes a through-hole being defined at least by the respective insertion portion and extending between longitudinally opposing ends of the respective part and the connection portions are configured to engage each other and are arranged such that the through-holes are in fluid communication with each other in a connected state, i.e. when the connection portions of the first part and of the second part are engaged.

### CROSS-REFERENCE TO RELATED APPLICATIONS

This application is the U.S. National Stage of PCT/EP2023/087467, filed on Dec. 21, 2023, which claims priority to PCT/EP2022/087899, filed on Dec. 27, 2022, the entire contents of which are incorporated herein by reference.

### TECHNICAL FIELD

The present disclosure relates to nerve conduit systems for connecting two nerve ends in an end-to-end manner. In particular, the present disclosure relates to nerve conduit systems supporting the repair of nerve lesions by facilitating proper placement and orientation of the nerve ends and enabling the connection of nerve ends, particularly nerve ends having different diameters.

### BACKGROUND

Upon injury, tissue damage may involve one or more nerve lesions of the peripheral nerve system resulting in a partial sensory loss and/or impaired motor skills. Such injuries involving nerve damage particularly occur in the upper extremities, such as a hand or finger of an individual, such that an individual may experience a loss e.g. in tactile or haptic feedback and/or may have difficulties in controlling fine motor skills in the injured region, if the nerve lesion is not treated properly.

Current treatments of nerve lesions include coaptation of the nerve ends by various suturing techniques so as to provide a connection between the respective nerve ends in a substantially tensionless manner. In case a more severe defect is present, wherein the nerve ends are not directly adjacent to each other, a reconstruction may be required to overcome a corresponding gap. A reconstruction may be provided e.g. by an autologous or allogenic nerve graft. Alternatively, a reconstruction may be performed by providing a tubular structure so as to provide a nerve guide. Such tubular structure may be provided e.g. by autologous or allogenic venous structures or by artificially manufactured nerve conduits made of a biocompatible material. The use of a tubular structure may furthermore facilitate the repair of the lesion irrespective of the presence of a gap, e.g., by providing further mechanical support and structural stability, providing a tensionless repair, reducing an inflammatory response to the site of the lesion, and/or limiting the extent of fibrous tissue development.

The tubular structures and, in particular, nerve conduits are generally formed as a substantially cylindrical shape extending in a longitudinal direction and defining an inner lumen or through hole from one end to the opposing end. The dimensioning and shape are chosen so as to be compatible with the affected microstructure of the injured nerve tissue and surrounding tissue. Accordingly, in order to treat a nerve lesion, a nerve end is inserted into the nerve conduit via one end of the nerve conduit and the corresponding other nerve end is inserted into the nerve conduit via the opposing end of the nerve conduit. Both ends may then be fixed or coupled to the nerve conduit by suturing techniques or by applying or depositing a medical adhesive, for example. Alternatively, nerve ends may be fixed or coupled to the nerve conduit by one system combining movable contact member and pressing members with the pressing member applying mechanical force to the contact member to push and move it in the radial direction of the nerve (WO2012133019). In the implanted state, the nerve conduit hence forms a nerve guide, wherein the continuous cylindrical shape of the nerve conduit and the inner lumen provide a directional path during neurogenesis.

A further potential complication resulting from a nerve lesion is the increased risk of a neuroma, which may develop at the terminal end of a sectioned nerve. Such neuromas may develop from disorganized axons growing beyond the severed epineural sheath at the end of a transected nerve, e.g. following severe trauma or surgery, wherein a sprouting of axons may form a bundle of disorganised and hyperexcitable nerve tissue that does not have the original nerve structure. Neuromas are typically associated with neuropathic pain, numbness and tingling and may also interfere with the rehabilitation, the functional recovery and a sensory deficit. Treatments or prevention of neuromas may be based on suturing the proximal end of a lesioned nerve to a distinct motor nerve of an intact newly denervated muscle. Such treatment may be particularly applied in situations where a large gap between the proximal and distal nerve end exists or wherein the distal nerve end is lost due to amputation or tissue removal. The suturing technique may at least partially restore muscle control while reducing the risk of neuroma development.

### SUMMARY

Starting from the known prior art there is a need to further facilitate the repair of nerve lesions.

According to the present disclosure it has been recognized that the insertion and attachment of nerve ends into respective ends of a nerve conduit may be difficult, particularly in situations wherein large gaps between the nerve ends need to be bridged by the nerve conduit. This has been found to be caused, on the one hand, by the limited in situ flexibility of the nerve conduit. Once one nerve end has been introduced and secured to the respective end of the nerve conduit, insertion and alignment of the other nerve end may be particularly impaired and further handling of the nerve conduit may be limited. On the other hand, the continuous structure of the nerve conduit and the absence of visual feedback renders it difficult for a surgeon to assess whether a respective nerve end has been properly inserted and is correctly held in place after attachment, for example by the application of a medical adhesive. Potential necessary modifications are hence generally not possible.

Furthermore, it has been found that differences in diameters of the respective nerve ends to be connected may be present, in particular in case of larger gaps and/or when connecting a proximal nerve end to a distinct nerve end, e.g. a distinct motor nerve, wherein the latter may have a considerably smaller or larger diameter. In order to provide a connection for such nerve ends exhibiting such differences in diameter, the dimensions of the nerve conduit need to be accordingly increased, which, however, is not desirable for the proper insertion and securing of the nerve end having the smaller diameter. In particular, this may require larger volumes of medical adhesive for proper securing of at least the nerve end having the smaller diameter, which further complicates the implementation and handling of the nerve conduit. The larger diameter and the large amount of medical adhesive may furthermore result in improper axonal growth, impairing restoration and potentially even inducing the development of a neuroma.

It is hence an object of the present disclosure to further improve and/or facilitate the repair of nerve lesions. In particular, it is an object of the present disclosure to facilitate proper securing and alignment of nerve ends to a nerve conduit. It may be a particular object of the present disclosure to facilitate such repair for nerve ends having different diameters.

Said object is achieved by the claims. Embodiments are depicted in the claims, the description, and the Figures.

Accordingly, a nerve conduit system for connecting two nerve ends in an end-to-end manner is provided, comprising a first part and a second part, each part comprising an insertion portion being adapted to receive one of the two nerve ends, and a connection portion, wherein each part comprises a through-hole being defined at least by the respective insertion portion and extending between longitudinally opposing ends of the respective part and wherein the connection portions are configured to engage each other and are arranged such that the through-holes are in fluid communication with each other in a connected state, i.e. when the connection portions of the first part and of the second part are engaged.

It is noted that when the first and second part are engaged, the connection portions do desirable not act as a pressing member applying a force in the radial direction of the nerve end. More particularly, the connection portion of the first part does not act as a pressing member on the connection portion of the second part applying a force in the radial direction of the nerve end and the connection portion of the second part does not act as a pressing member on the connection portion of the first part applying a force in the radial direction of the nerve end.

In other words, the nerve conduit system of the present disclosure comprises or consists of two separate (individual) parts (the first part and the second part), which can be connected to each other (by their connection portions) in order to bring the two nerve ends closer, and in extreme case to connect the two nerve ends. Each part and respective insertion portion hence receives a respective one of the two nerve ends to be connected, such that the nerve ends are accommodated in the through-hole of the respective part. By the connection and the fluid communication of the through-holes, the nerve ends may hence face each other in an end-to-end manner.

As used herein, connecting nerve ends in an “end-to-end manner” includes the direct connection of (two) nerve ends, i.e. without a gap in between the nerve ends, as well as the connection of (two) nerve ends with a gap or longitudinal distance of up to about 20 mm, up to about 15 mm, up to about 10 mm, or up to about 5 mm. In some embodiments, the (two) nerve ends are hence directly connected, i.e. without a gap in between the nerve ends. In other embodiments, the (two) nerve ends are connected with a gap or longitudinal distance of up to about 20 mm, up to about 15 mm, up to about 10 mm, or up to about 5 mm between the (two) nerve ends.

The provision of the two individual parts has the aspect that the handling of the nerve conduit system may be particularly facilitated, since each nerve end may be individually fixed or attached to the respective (first or second) part before connecting both parts by the corresponding connection portions. Thereby, handling flexibility for the surgeon is improved. This furthermore provides that the insertion of the respective nerve end into the corresponding insertion portion is facilitated while it may be ensured that a medical adhesive or other techniques used for securing the respective nerve end to the corresponding (first or second) part has been properly applied. The nerve ends may hence optionally be fixed or attached to the respective part before they are connected to each other.

Additionally, the two individual parts desirably enable the selection of each part according to the specific size (diameter) of each nerve end. This is particularly desirable for the connection of nerve ends having different diameters, because each part of the nerve conduit system can be selected according to the distinct size of the nerve end. In particular, the respective parts may comprise an insertion portion defining a portion of the through-hole and having an inner diameter corresponding to the respective nerve end to be inserted into the insertion portion and accommodated within the through-hole. In contrast thereto, conventional single-part nerve conduits are usually chosen according to the nerve end with the larger diameter, such that the selected nerve conduit is usually too large for the nerve end with the smaller diameter and requires special fixation.

The first part may comprise an insertion portion and a corresponding portion of the through-hole having an inner diameter adapted for a nerve end having a larger diameter (compared with the (other) nerve end to be connected therewith). In accordance, the second part may comprise an insertion portion and a corresponding portion of the through-hole having an inner diameter adapted for a nerve end having the smaller diameter (compared with the (other) nerve end to be connected therewith). The configuration of the first and second part may hence be optimized for the particular nerve end diameters. By the connection portions and the fluidly connected through-hole, the nerve ends may then be connected to each other in an end-to-end manner. In embodiments, the connection portions are standardized for the various dimensions of the insertion portions of the respective parts.

Accordingly, the nerve conduit system with the two combinable yet individual first and second parts enables that the insertion portions of the respective parts may each comprise an inner through-hole having different diameters and/or cross-sectional areas. For example, a mismatch in diameter of the nerve ends between 1.1 fold and 10 fold, in particular more than two-fold, may be bridged by corresponding adaptation of the respective first and second parts of the nerve conduit system. For example, at least the insertion portion of one part may be configured to receive a nerve end having a diameter of between 1 mm to 12 mm (e.g. 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, 10.5 mm, 11 mm, 11.5 mm, or 12 mm), while the insertion portion of the other part may be configured to receive a nerve end having a diameter of between 1 mm and 12 mm (e.g. 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, 10.5 mm, 11 mm, 11.5 mm, or 12 mm).

It must however be understood that the two nerve ends to be connected may have the same diameter, the first and second insertion portions and corresponding portion of the through-hole are then adapted accordingly.

Another aspect of having individual parts for the respective nerve ends is that the nerve ends may be observed or inspected after insertion into the respective insertion portion (into the through-hole). In particular, the respective nerve end may be received in the through-hole via (in) the corresponding insertion portion. It may extend through the through-hole from one longitudinal end towards the opposing longitudinal end (of the insertion portion). The nerve end may typically be inserted in such manner that the nerve end does not extend beyond the insertion portion, but terminates within the through-hole at and/or in the region of the junction between insertion portion and an adjacent portion. Thereby, the nerve end may be observed via the opposing longitudinal end of the through-hole. Accordingly, proper insertion may be monitored prior to the application of a medical adhesive and/or connection of the parts.

The nerve conduit parts may generally comprise a substantially tubular shape with a through-hole with longitudinally opposing openings and one or more (side)walls. Thereby, a continuous channel or through-hole is provided in each of the individual parts and, in particular, also when the connection portions of the first part and of the second part are engaged, i.e. in the connected state (by connecting the (single) through-holes of the individual parts to form a continuous (single) through hole in the connected state). While the nerve conduit parts may have a substantially tubular shape, the diameter of said tubular shape may vary along the longitudinal direction. In the connected state, i.e. when the connection portions of the first part and of the second part are engaged, the parts may thus form a longitudinally extending elongated body, wherein the respective through-holes are fluidically coupled to each other, e.g. by being in a directly adjacent arrangement or by accommodating an end portion of the through-hole of one part and not used for insertion of the respective nerve end in the through-hole of the other part.

According to another embodiment, the tubular shape may include structures with particular geometries or surface irregularities shaped to form retention surfaces, such as protrusions, slits, threads, grooves or holes on the outer surface, more particularly on the outer surface of the insertion portions. In particular, such external structures may (i) increase the contact between the surface of the insertion portion and the medical adhesive; and (ii) create anchor points for the medical adhesive. Such external structures are disclosed for example in patent application WO2022229207 the content of which is incorporated by reference in its entirety.

In embodiments, the cross-section of the through-hole is of circular or ellipsoid shape. A tubular or cylindrical shape structure may substantially correspond to the shape of the through-hole, such that a continuous guiding structure for tissue repair or support (protection), e.g. neurogenesis or nerve growth, is typically provided, without providing an undesirable biasing. It may provide sufficient structural stability and may prevent sharp bends or kinking during tissue movement, i.e. compression or extension or nerve growth. The tubular shape may accordingly also provide that a homogeneous structure is provided, which reacts in a predefined manner along the entire longitudinal extension, in particular when forces act upon a portion of the nerve conduit system, e.g. upon impact.

The through-hole of each part may hence be continuous and be delimited by the end faces of openings at the longitudinally opposing ends. In some embodiments, for one or both parts of the system, in particularly for the second part (but not for the first part), the through-hole may be defined exclusively by the insertion portion. In other embodiments, for one or both parts of the system, particularly for the first part (but not for the second part), the through-hole may be defined by the insertion portion and, additionally, also by portions of the respective part being connected to and/or extending from the respective insertion portion, such as the connection portion and/or the transition portion.

The insertion portion may define a proximal end and a longitudinally opposing distal end of the corresponding portion of the through-hole. That is, the proximal end (or proximal opening) may be used to insert the respective nerve end into the insertion portion and corresponding portion of the through-hole. In embodiments, the distal end may be arranged such that the respective nerve end does not exit or protrude from said end. The distal ends of the insertion portions of the first and second part may face each other in the connected state, i.e. when the connection portions of the first part and of the second part are engaged. Further portions of the respective part may extend from the distal end, e.g. to facilitate connection and/or to facilitate guidance to axonal growth of one of the nerve ends towards the other nerve end to be connected, limiting the risk of neuroma while supporting nerve regeneration and recovery.

In embodiments, at least one part, or the first part and the second part, further comprises a transition portion arranged or extending between the respective insertion portion and the respective connection portion of said part. The transition portion may extend from the insertion portion towards the connection portion and vice versa so as to form a bridging portion. The extension in this regard is to be understood as a longitudinal and/or radial extension. The provision of such transition portion is particularly desirable if the diameters of the insertion portion and of the connection portion of the same part differ from each other. In embodiments, the diameter of the insertion portion of one part, e.g. the first part, may be larger than the diameter of the respective connection portion of said part. Additionally or alternatively, the other part, e.g. the second part, may comprises a connection portion having a diameter being larger than the diameter of the respective insertion portion. In particular, the transition portion “bridges” the different diameters of insertion and connection portion in the same part, such that the (first and/or second) part can be integrally formed, without abrupt or sharp-edged steps (in its outer shape).

Thus, the transition portion has a substantially tapered shape or conical shape, e.g. as seen in a longitudinal section. Thereby, a gradual transitioning from a diameter of the insertion portion to the diameter of the connection portion may be provided, in a stepless manner. In some embodiments, the transition portion thereby comprises a substantially circular cross-section.

In some embodiments, the outer surface of the transition portion and/or the connection portion may furthermore facilitate the handling of the respective part, e.g. by forming an outer gripping and/or support surface towards the connection portion. This may be particularly the case, when the transition portion comprises a substantially tapered shape or otherwise gradually transitioning shape.

In some embodiments, the transition portion of the second part may be provided to connect and arrange the respective connection portion outside of the insertion portion, e.g. to attach the insertion portion to the connection portion having a larger diameter, in a surrounding and concentric manner. In some embodiments, the transition portion of the first part may be provided to attach the insertion portion to the connection portion, in an adjacent and radially inwardly or outwardly extending manner. In some embodiments, in (at least) one part, particularly in the first part, the connection portion and, optionally, the transition portion may furthermore define a respective portion of the through-hole (in addition to the insertion portion defining (another) portion of the (same) through-hole). This may be achieved in a configuration, wherein the insertion portion and the connection portion of (at least) one part, particularly the first part, are arranged adjacent to each other in longitudinal direction, either directly or with the transition portion in between. In such configuration the shape of the through-hole defined by the transition portion comprises a shape corresponding to the transition portion as a whole, such that the outer diameter and the inner diameter extend substantially in parallel. The shape of the transition portion may not only provide that a transition between different diameters of the through-hole is established, but may also facilitate the handling of the respective part, e.g. by forming an outer gripping and/or support surface (even if no transition between different diameters of the insertion and connection portion is required, in particular if said diameters are the same).

In some embodiments, in (at least) one part, particularly in the second part, the transition portion and the connection portion may be arranged in such manner that they do not define a respective portion of the through-hole. For example, the transition and/or the connection portion may at least partially surround the insertion portion as described below.

In some embodiments, the insertion portion and/or the connection portion of at least one part, particularly of each part, may furthermore comprise a substantially cylindrical (or tubular) shape. As described above, this may improve overall structural stability and handling. Furthermore, such shape provides that the radial dimensioning may be optimized in accordance with the diameter of the respective nerve conduit. In addition, a cylindrical shape reduces or avoids the occurrence of sharp edges and/or inadvertent clamping or compression of the surrounding tissue in the implanted state of the nerve conduit system.

As described above, the insertion portion of the first and/or second part may have a substantially tubular shape. Accordingly, the insertion portion of the first and/or second part may extend along a longitudinal axis. Moreover, the connection portion of the first and/or second part may have a substantially tubular shape. Accordingly, the connection portion of the first and/or second part may extend along a longitudinal axis. In some embodiments, the longitudinal axes of the insertion portion and of the connection portion of the same part correspond to each other (e.g., it may be the same or the longitudinal axis of the insertion portion is a longitudinal extension of the longitudinal axis of the connection portion of the same part). In other words, for each part the insertion portion, the connection portion and, optionally, the transition portion may extend along the same longitudinal axis. In the connected state, i.e. when the connection portions of the first part and of the second part are engaged, it is desirable that the insertion portions of the first and the second parts extend along the same longitudinal axis. Likewise, it is desirable that the connection portions of the first and the second parts extend along the same longitudinal axis. In some embodiments, the insertion portion, the connection portion and, optionally, the transition portion of the first and the second part extend along the same longitudinal axis when the connection portions of the first part and of the second part are engaged, i.e. in the connected state.

In some embodiments, the connection portion of (at least) one part, particularly the second part, may at least partially surround the insertion portion of said part. This may be either directly or, desirably, having a transition portion arranged between the insertion portion and the connection portion. In some embodiments, the transition portion provides for a spacing between the insertion portion and the connection portion (the latter surrounding the insertion portion at least partially and, in some embodiments, particularly entirely). In some embodiments, the transition portion is arranged such that the transition portion may (also) at least partially surround the insertion portion. This “surrounding” configuration may be particularly desirable, if the insertion portion of one part, particularly the second part, is configured for a nerve end having a smaller diameter compared with the nerve end to be inserted into (and accommodated by) the insertion portion of the other part. Thereby, the longitudinal dimensioning of said part as a whole may be reduced while maintaining connectability of the respective connection portions. In particular, the insertion portion and the connection portion of at least one part may be arranged in a concentric manner. In a configuration, wherein the connection portion partially or fully surrounds the respective insertion portion, the connection portion does not define a corresponding portion of the through-hole.

In some embodiments, the insertion portion and the connection portion of (at least) one part, particularly the first part, may be arranged adjacent to each other in longitudinal direction. This may be either directly or having a transition portion arranged between the insertion portion and the connection portion. Such subsequent configuration in the longitudinal direction, which is in contrast to a concentric arrangement, might be particularly desirable, if the insertion portion of the respective part comprises substantially the same diameter or a larger diameter compared with the connection portion. In such configuration, the connection portion defines a corresponding portion of the through-hole. Accordingly, it may be similarly dimensioned or smaller dimensioned compared with the portion of the through-hole defined by the insertion portion. In case a difference in diameter between the insertion portion and the connection portion is to be bridged, a transition portion accommodating for said difference may be arranged between the connection portion and the insertion portion, wherein the transition portion also defines a corresponding portion of the through-hole.

According to one embodiment, both parts, i.e. the first and the second part, may have the same (surrounding or subsequent) configuration with regard to the arrangement of the connection and insertion portion relative to each other, i.e. (i) in both parts the connection portion may at least partially surround the insertion portion of said part (“surrounding configuration”), or (ii) in both parts the insertion portion and the connection portion may be arranged adjacent to each other in longitudinal direction (“subsequent” configuration). According to an embodiment, in only one of the parts, particularly the first part the insertion portion and the connection portion may be arranged adjacent to each other in longitudinal direction (“subsequent” configuration), while in the other part, particularly the second part, the connection portion may at least partially surround the insertion portion of said part (“surrounding configuration”).

To facilitate the connection of the nerve ends in an end-to-end manner, the connection portions may be configured such that the insertion portions are arranged at longitudinally opposing ends of the nerve conduit system when the connection portions of the first part and of the second part are engaged, i.e. in the connected state. Depending on the required configuration, any optionally available further portions defining the through-hole, e.g. a transition portion, are hence arranged between the insertion portions when the connection portions of the first part and of the second part are engaged, i.e. in the connected state. The arrangement at longitudinally opposing ends may be such that openings of the portion of the through-hole being defined by the respective insertion portions (which are configured to receive and insert the respective nerve end into the respective insertion portion, i.e. configured as a nerve end inlet into the insertion portion) face away from each other when the connection portions of the first part and of the second part are engaged.

In some embodiments, the (single) through-holes of the first and second part define a single longitudinal axis of the nerve conduit system when the connection portions of the first part and of the second part are engaged, i.e. in the connected state. By inserting the nerve ends into the respective insertion portions, the terminal end faces of the nerve ends may hence be directly facing each other and radial offsets or angles in the end-to-end connection provided by the nerve conduit system may be avoided to facilitate an even more efficient and direct repair of the lesioned nerve. In some embodiments, the through-holes are arranged and aligned in a concentric or adjacent manner in the connected state such that the nerve ends to be connected may accordingly be connected in an efficient manner.

The through-holes of the two parts may be arranged in a subsequent and adjacent manner in the longitudinal direction. In some embodiments, the connection portions are configured such that the through-hole of one part, in particular the second part, is at least partially accommodated within or close to the through-hole of the other part, e.g. the first part, when the connection portions of the first part and of the second part are engaged, i.e. in the connected state.

By accommodating the portion of the through-hole, e.g. of the second part, in the through-hole of the first part, the overall longitudinal dimensions of the nerve conduit system may be reduced. Furthermore, such configuration may improve structural stability of the nerve conduit system and/or sealing of the nerve ends to be connected towards the exterior of the nerve conduit system, e.g. by a corresponding configuration of at least the respective connection portions. In particular, the connection portion of the first part may define a corresponding portion of the through-hole of the first part. In some embodiments, the connection portion of the first part is adapted to accommodate the insertion portion of the second part and its corresponding portion of the through-hole.

The accommodation within or close to the through-hole of the first part may furthermore facilitate regeneration of a lesioned nerve end by ensuring that the respective nerve ends to be connected are brought into close(r) proximity to each other. Axonal growth e.g. from the nerve end being accommodated in the through-hole of the first portion towards the nerve end being accommodated in the through-hole of the second portion may hence be supported.

In some embodiments, such accommodation within the through-hole of a respective part, e.g. the first part, may be particularly desirable, if said part is configured to accommodate a respective nerve end having a larger diameter compared with the nerve end to be accommodated in the through-hole of the other part. Accordingly, it is particularly desirable if the connection portion of said part, e.g. the first part, is arranged in a subsequent or adjacent manner relative to the respective insertion portion, e.g. via a respective transition portion, wherein the connection portion and corresponding portion of the through-hole of said part may be dimensioned larger than the insertion portion of the other part, e.g. the second part.

The connection portions of the respective parts, however, may have predefined dimensions that are independent of the dimensioning of the insertion portion of the respective part. The connection portions may hence be configured as standardized connection portions for a variety of dimensions of the insertion portions, e.g. in a modular kit of parts.

It is to be understood that the above described embodiment may be equally applicable for a configuration, wherein e.g. the second part is configured to receive a nerve end having substantially the same diameter or a larger diameter compared with the nerve end to be received by the first part. In either configuration, the connection portion of at least one of the parts is arranged in a subsequent or adjacent manner relative to the respective insertion portion, e.g. via a respective transition portion, wherein the connection portion and corresponding portion of the through-hole of said part is dimensioned larger than the insertion portion of the other part, e.g. the second part.

In the connected state, i.e. when the connection portions of the first part and of the second part are engaged, the nerve ends are accommodated (only) within the respective insertion portions. Thereby, the nerve ends can be connected in an end-to-end manner without extending into the insertion portion of the respective other part. The connection portion defining the through-hole accommodating the corresponding through-hole of the other part, e.g. the corresponding insertion portion of the other part, may be configured to only accommodate a portion of said insertion portion of the other part. A predefined gap between the end faces of the respective nerve ends may be provided in the accommodated and connected state, i.e. when the connection portions of the first part and of the second part are engaged.

Said connection portion may extend from a respective transition portion, which extends between the insertion portion and the connection portion to provide a bridging portion between different diameters. In such configuration, the transition portion may, alternatively or in addition, also at least partly provide said predefined gap.

Accordingly, the overall dimensions of the nerve conduit system may be adapted to the requirements corresponding to the respective nerve ends to be connected, rendering the need of oversized conduits substantially obsolete.

In some embodiments, the through-hole of the first part may furthermore comprise a longitudinal end portion, which is arranged so as to oppose a longitudinal end portion being configured to receive the respective nerve end and which comprises a cross-sectional area of the through-hole that is gradually increased in a longitudinal direction away from the insertion portion. For example, the local increase in diameter of the through-hole may be provided at a distal longitudinal end portion of the first part defined by the respective connection portion and/or a respective free longitudinal end portion.

Accordingly, the increase in diameter is not intended to facilitate insertion of the respective nerve end, but may e.g. facilitate the connection of the first part with the second part, in particular, when the corresponding connection portion of the first part extends from the distal end of the insertion portion and is adapted to accommodate the connection portion of the second part therein.

In such configuration, the connection portion of the second part engages the connection portion of the first part at an outer surface of the connection portion of the first part. In other words, the connection portion of the first part is formed by a circumferential wall, wherein, e.g., a circumferential wall of the second part may engage the outer circumference of the connection portion of the first part. In some embodiments, if the first part is provided with a transition portion, the transition portion of the first part is configured to at least partially receive and accommodate a radially expanding end portion of the insertion portion of the second part when the connection portions of the first part and of the second part are engaged, i.e. in the connected state. Accordingly, the second part may comprise at least one radially expanding end portion(s) of the insertion portion. In some embodiments, the radially expanding end portion of the insertion portion is (only) at the end receiving the nerve end (nerve end “inlet” into the insertion portion). In some embodiments, the radially expanding end portion of the insertion portion is (only) at the end (longitudinally) opposing the end receiving the nerve end. Accordingly, the radially expanding end portion may e.g. extend from a distal end of the insertion portion, i.e. an end not being configured to insert the respective nerve end. In some embodiments, both ends of the insertion portion of the second part may have a radially expanding end portion.

In some embodiments, the radially expanding end portion (at the end of the insertion portion opposing the nerve inlet end) is configured such that the nerve end being accommodated in the through-hole does not extend through or beyond said end portion. Accordingly, the radially expanding end portion may delimit the end face of the respective nerve end. Thereby, a spacing or gap may be provided relative to the nerve end being accommodated in the portion of the through-hole being defined by the corresponding insertion portion. Inadvertent extension of the nerve ends into the insertion portion of the respective other part may hence be avoided, such that the nerve ends may be accommodated in a substantially tensionless manner.

Furthermore, the radial expansion of the end portion, e.g. having a conical shape, may provide guidance for axonal nerve growth or regeneration from the nerve end being accommodated in the through-hole of the first part towards the nerve end being accommodated in the through-hole of the second part, i.e. a target nerve end. The radially increased end portion may e.g. abut or may have a predefined spacing to the adjacent nerve end and/or may be dimensioned to prevent growth along the outer circumference of the insertion portion of the second part, e.g. if the second part is dimensioned for a nerve end having a smaller diameter.

Alternatively, the radially expanding end portion may also be accommodated within the portion of the through-hole being defined by the connection portion of the first part when the connection portions of the first part and of the second part are engaged, i.e. in the connected state. Depending on the configuration of the first part, a longitudinal end face of the radially expanding end portion may e.g. be positioned at the junction of the connection portion and the adjacent transition portion or insertion portion. The radially expanding end face may e.g. radially abut the through-hole of the first part in a frictionless manner or may be dimensioned to define a predefined gap between said through-hole and the radially expanding end portion. Thereby, a guiding surface and/or longitudinal delimitation for the nerve end being accommodated in the through-hole of the first part may be provided. Such guiding surface provided by the radially expanding end surface may e.g. be substantially contiguous and/or flush with the adjacent transition portion or insertion portion of the first part.

In some embodiments, the insertion portion of the second part and/or an optionally present radially expanding end portion thereof extend beyond the respective connection portion in the longitudinal direction, in particular in a distal direction. Such configuration facilitates that the distal end portion of the insertion portion may extend into the through-hole of the first portion, typically beyond the connection portion of the first part. Furthermore, such extension into the through-hole of the first part and towards the nerve end received in the insertion portion of the first part may minimize the gap between the respective nerve ends that are connected in an end-to-end manner. Accordingly, an extension from the insertion portion may provide that a larger portion of the insertion portion and corresponding through-hole of the second part may be received and accommodated by the first part, i.e. within the respective through-hole, which may be desirable to bring the terminal ends of the nerve ends into proximity with each other.

At least a portion of the through-hole, which is defined by (at least a portion of) the insertion portion of the first and/or second part, may be defined by a tubular shaped wall defining a substantially constant inner diameter. The constant inner diameter may be desirable in view of the diameter and/or morphology of the nerve end to be accommodated therein. Furthermore, this may facilitate insertion of the respective nerve end by avoiding constrictions of the through-hole, which may be undesirable. The through-hole of the second part may be substantially defined by the respective insertion portion (only). Accordingly, in the second part the insertion portion (only) substantially defines the longitudinal extension of through-hole (while the connection portion and, optionally, the transition portion, of the second part may not define the through-hole, when arranged in the “surrounding” configuration, i.e. said portion(s) surrounding the insertion portion). In some embodiments, the connection portion of the second part is hence configured and arranged so as not to define a portion of the through-hole, which may e.g. be provided in a configuration, wherein the connection portion is arranged around the insertion portion.

In some embodiments, the through-hole of the first part may also be substantially defined by the respective insertion portion, but is in addition substantially defined by the respective connection portion and, optionally, a transition portion. In the connected state, i.e. when the connection portions of the first part and of the second part are engaged, of the nerve conduit system, a through-hole of the system, i.e. a (single) continuous through hole extending between the first part and the second part, may then be substantially formed by the respective insertion portions of the two parts (only). Thereby, the nerve ends being accommodated in the respective insertion portions are hence connected, or at least brought into proximity, to each other via said through-hole.

The connection portion of the second part may extend from a longitudinal end portion of the respective insertion portion being configured to receive and/or insert the respective nerve end, e.g. from an inlet of the respective insertion portion. Alternatively, the connection portion of the second part may extend from the insertion portion with a longitudinal offset to a longitudinal end portion of the respective insertion portion being configured to receive and/or insert the respective nerve end. For example, the connection portion may extend from a proximal end of the insertion portion or from a position between the proximal end and a distal end of the insertion portion. In general, the connection portion of the second part may either directly extend from the insertion portion or indirectly, wherein a transition portion may be arranged between connection and insertion portion of the second part.

In the surrounding configuration as described above, the connection portion extends in the longitudinal direction towards the distal end of the insertion portion along an outer surface of the insertion portion. In some embodiments, a wall of the connection portion surrounds a wall of the insertion portion. Thereby, in some embodiments, a substantially equal distance is provided between the wall of the connection portion and the wall of the insertion portion (in longitudinal and/or in radial direction). In some embodiments, a radial spacing between the connection portion and the insertion portion is provided by a transition portion. Thereby, the transition portion may be configured to attach the connection portion having a larger outer diameter with the insertion portion having a smaller diameter.

Accordingly, the connection portion may be formed e.g. as a circumferential wall. The connection portion may be connected at one end to a wall defining the insertion portion and corresponding portion of the through-hole, in particular via a transition portion as described above. The connection portion of the second part may comprise a “free” end or a protrusion (in the longitudinal direction), which is configured to initiate the connection with the first part. That is, said free end or protrusion may be providing the first contact with the connection portion of the other (first) part during the connection of both parts.

In particular, an offset of the connection portion of the second part to e.g. a proximal end of the insertion portion of said second part may be desirable, for example when the overall longitudinal extension of the second part is larger to bridge a corresponding larger nerve end gap. In such case, a longitudinal offset to the insertion portion may be desirable for improved structural stability and/or handling of the connection portion of the second part.

In some embodiments, the connection portion of the first part is arranged adjacent to a respective transition portion or a longitudinal end portion of the respective insertion portion opposing a longitudinal end portion of the respective insertion portion being configured to receive and/or insert the respective nerve end.

In some embodiments, the connection portion of the first part may be formed by a single circumferential wall defining a corresponding portion of the through-hole. Said wall may furthermore be configured to accommodate and engage the connection portion of the second part. The connection portion of the second part may be formed as a circumferential wall.

Accordingly, the connection portion of the first part may be formed by a single circumferential wall, wherein the single wall accommodates the connection portion of the second part at the inner surface or outer surface of said wall. Hence, the connection portion of the second part may be received within the first part or at the outer surface of the first part, e.g. by a slidable arrangement, wherein the wall of the first part may form a guiding surface and engages the connection portion of the second part, which is also formed as a circumferential wall. In such embodiment, the connection portion of the first part defines a corresponding portion of the through-hole, which may particularly accommodate at least a portion of the through-hole of the second part.

The provision of a single circumferential wall facilitates the connection, since a correct relative orientation both in radial and longitudinal direction of the two parts is simplified. Furthermore, the accommodation of the wall of the connection part of the second part into the through-hole defined by the wall of the connection portion of the first part or along said wall of the connection portion, e.g. by sliding arrangement, provides the surgeon with haptic feedback regarding the coupling and may provide a progressing engagement depending on the length of insertion.

In some embodiments, the connection portion of the first part is formed as a double-wall portion, e.g. two circumferential walls being radially spaced apart so as to define a gap in between. In some embodiments, the two circumferential walls of said connection portion are arranged in a substantially concentrical manner. The two circumferential walls may be connected to each other at one (longitudinal) end, which is adjacent to the transition or insertion portion of the first part. In some embodiments, the (longitudinal) end opposing thereto is a “free” end, which is configured to accommodate and engage the connection portion of the second part. Accordingly, in some embodiments, the connection portion of the second part, a single (circumferential) wall, may be received within the gap provided between the two walls of the connection portion of the first part. It is understood that said gap between the two walls of the connection portion of the first part is typically dimensioned such that it can receive/accommodate the connection portion of the second part, a single (circumferential) wall.

In other words, the connection portion of the first part may be formed by a circumferential inner and outer wall defining a longitudinally extending groove between said walls (also referred to as “gap” herein above). In particular, the groove is configured to accommodate the connection portion of the second part when the connection portions of the first part and of the second part are engaged, i.e. in the connected state. The inner wall and outer wall of the connection portion are hence radially spaced apart from each other. In some embodiments, the groove may also be formed within a single (thicker) wall portion (instead of respective wall portions), hence defining an inner and outer wall element or portion.

In some embodiments, the inner wall and outer wall are formed so as to extend from a longitudinal end of the connection portion which is adjacent to the transition portion (or the insertion portion, if no transition portion is present). At the other end (in longitudinal direction) of the connection portion, a free end of the inner and outer wall is provided, which is configured to receive the connection portion of the second part. Said free end usually faces away from the respective insertion portion and/or from a corresponding opening to the through-hole configured as an inlet to receive the nerve end. In particular, the inner and outer wall are connected to each other at the (longitudinal) end of the connection portion, which is adjacent to the transition portion (or to the insertion portion). In some embodiments, the other longitudinal ends of the walls are free, forming a respective protrusion and a corresponding seat (or groove/gap) for (receiving) the connection portion of the second part. By the groove or double wall, the connection portion of the second part, which is, in some embodiments, also formed as a circumferential wall, may be inserted into the connection portion of the first part, i.e. into the groove, so as to engage the outer wall and/or inner wall, e.g. by interference fit.

The outer wall may furthermore comprise a radial flare or extension at its free end, e.g. in the form of a funnel, chamfer, or bevel. Thereby, in some embodiments, the radius of the outer wall of the connection part of the first part may be increased at its free end as compared to its other end (and as compared to the major part of the connection part, where the inner and outer wall extend in a substantially concentrical manner around the same longitudinal axis as the connection part of the second part and/or the respective section of the through-hole). This radial flare may facilitate the insertion of the connection portion of the second part. Such flare may furthermore be used to release an intermittent coupling of the two parts, e.g. by facilitating a spacing between the outer wall and a received portion of the connection portion of the second part.

As described above, each connection portion may comprise at least one circumferential wall in order to facilitate the connection of the two parts via the corresponding connection portions. In some embodiments, the circumferential walls are configured to engage and radially bias each other and/or are dimensioned and configured to be coupled to each other by interference fit when the connection portions of the first part and of the second part are engaged, i.e. in the connected state.

According to one embodiment, the outer diameter of the circumferential wall of the connection portion of the second part may be adapted to (e.g., substantially correspond to or being slightly smaller than) the inner diameter of the (outer) circumferential wall of the connection portion of the first part, such that a press-fitted engagement of said circumferential walls is enabled. According to some embodiments, the inner diameter of the circumferential wall of the connection portion of the second part may be adapted to (e.g., substantially correspond to or being slightly bigger than) the outer diameter of the (inner) circumferential wall of the connection portion of the first part, such that a press-fitted engagement of said circumferential walls is enabled. Usually, a certain amount of tolerance and/or flexibility is provided with regard to said diameters to enable a press-fitted engagement of the circumferential walls. For example, a difference in said diameters of the circumferential walls may correspond to between about 30 percent and 90 percent of the wall thickness or between 40 percent to 60 percent of the wall thickness. One of the circumferential walls may furthermore comprise a larger thickness, such that the radial biasing force towards the circumferential wall having the smaller thickness may be improved, e.g. to facilitate a biasing force in the radially outward direction.

Furthermore, the circumferential walls may be dimensioned such that the wall of the connection portion of the second part is slidably accommodated by at least one wall of the connection portion of the first part. Accordingly, the circumferential wall of the first part may provide a guiding surface facilitating the connection and engagement of the two parts. Thereby, the circumferential wall of the second part may be received at the outer surface or inner surface of the circumferential wall of the first part.

The slidable arrangement may furthermore provide that an initial hold is provided prior to a potential fixation of the two parts, e.g. by application of a medical adhesive or mechanical fixation when the connection portions of the first part and of the second part are engaged, i.e. in the connected state. A guiding surface and slidable arrangement furthermore facilitate correct orientation and alignment of the nerve conduit parts and the nerve ends received therein.

Alternatively, or in addition to a press-fit engagement, the parts may also be secured or fixed to each other by positive locking. Such positive locking may reduce the need for a medical adhesive and/or may facilitate the coupling of the parts and connection of the nerve ends to each other, potentially reducing the occurrence of a loosened engagement.

Accordingly, one of the connection portions may comprise at least one radial protrusion and the other connection portion may comprise at least one slit or groove configured to receive said protrusion and at least longitudinally secure the second part to the first part when the connection portions of the first part and of the second part are engaged, i.e. in the connected state. In some embodiments, the at least one radial protrusion and the at least one slit are configured to be in snap fit engagement in the connected state, i.e. when the connection portions of the first part and of the second part are engaged. It is to be understood that at least one protrusion or slit may hence refer to more than one protrusion or slit or groove, i.e. 2, 3, 4, 5, 6 or more, yet may also refer to a single protrusion or slit or groove. In general, the number of protrusions and the number of slits or grooves may be the same or different.

By providing the positive locking, a predefined connected state may be provided, such that a required or predefined distance or proximity between the nerve ends in the accommodated state may be ensured. Furthermore, such positive locking mechanism and, in particular, the snap fit engagement, provides the surgeon with a haptic feedback, confirming that the parts are properly connected to each other. Even if a medical adhesive is applied, e.g. for redundancy or particular treatment requirements, the parts are not required to be manually held together during the application and/or curing of the medical adhesive, since the positive locking ensures that the parts may be maintained in a predefined connected state, i.e. when the connection portions of the first part and of the second part are engaged.

In some embodiments, the at least one protrusion or the at least one slit of the connection portion of the first part is positioned at an inner or an outer surface of the circumferential wall of said connection portion. Alternatively, or in addition, the at least one protrusion or the at least one slit of the connection portion of the second part may be positioned at the opposite (i.e., outer or inner) surface of the circumferential wall of said connection portion. The at least one protrusion or the at least one slit of the connection portion of the first part may e.g. be present on a connection portion defined by single circumferential wall or may be arranged within a groove defined by a double circumferential wall defining the connection portion of the first part.

In particular, the circumferential wall of the connection portion of the second part may hence be received within or accommodated by the circumferential wall of the connection portion of the first part, i.e. being inserted inside a single circumferential wall or into a groove defined by the connection portion of the first part. In the connected state, i.e. when the connection portions of the first part and of the second part are engaged, the interfaces at the connection portion of the second part may hence engage the corresponding interfaces at the connection portion of the first part.

The at least one slit may comprise a larger circumferential extension than the at least one protrusion. This allows for some flexibility in the orientation of the parts relative to each other in the circumferential direction. Accordingly, the larger extension of the slit allows a corresponding movement or rotation of the parts corresponding to the difference in circumferential extension. This may be particularly desirable for situations, wherein the surgeon determines that a relative rotation may be beneficial for the treatment after initially securing the parts to each other. Furthermore, this may provide some level of natural motion at the implanted site and may e.g. absorb any torque forces potentially impairing the regeneration of the nerve. To prevent such further rotation after proper alignment, a medical adhesive may also be additionally applied. As described above, the positive locking provides that the parts are at least secured to each other in the longitudinal direction during application and/or curing of the medical adhesive.

In some embodiments, the nerve conduit system comprises a plurality of protrusions and a plurality of slits. In some embodiments, the number of slits and protrusions is equal. The protrusions and slits may be equally spaced apart in a circumferential direction.

In some embodiments, the nerve conduit parts comprise at least two slits and/or at least two protrusions. The plurality of protrusions and slits may provide an improved securing of the nerve conduit parts to each other and facilitates proper orientation and alignment for a surgeon during coupling of said parts. For example, the nerve conduit system may comprise between 2 and 12 protrusions and slits or 6 to 10 protrusions and slits. Furthermore, the slits may also be provided as two rows of slits, wherein the rows are longitudinally spaced apart from each other and wherein the slits of each row are provided along a single linear line in the circumferential direction. Thereby, a surgeon may choose between two different predefined gaps between the adjacent nerve ends in the coupled state and may select a required gap based on the clinical status of the nerve ends and/or the required treatment.

Instead of one or more slits, the nerve conduit system may also comprise one or more recesses configured to receive a corresponding protrusion. In such case, the protrusion will not extend through or beyond the receiving recess, as may be the case for a slit, but is accommodated, seated or held by the recess, e.g. in a form-fitting manner.

To facilitate the insertion of a nerve end into the respective insertion portion, the cross-sectional area of the through-hole of the insertion portion of the first part and/or the second part may be gradually increased at a longitudinal end portion for receiving the nerve end and in a direction facing toward the end receiving the nerve end (nerve inlet). In other words, an increase of the diameter of the through-hole along a longitudinal direction may be provided at the level or end face of the insertion portion where the respective nerve end is to be inserted into the through-hole, e.g. at an inlet of the respective insertion portion.

This may be particularly desirable for a nerve conduit part dimensioned to accommodate a nerve end having a smaller diameter, which may render it difficult to insert the corresponding nerve end into the opening. For nerve ends having a larger diameter, insertion into the accordingly dimensioned insertion portion may be less difficult. However, also in such case, an increased diameter or opening may facilitate the insertion and/or facilitates the application of a medical adhesive for securing the nerve end to the respective insertion portion.

In some embodiments, the insertion portion and, in particular, the corresponding portion having the increased cross-sectional area of the through-hole have a rotationally symmetric shape along a longitudinal axis defined by a wall. In particular, said shape may be a substantially U-shape, sigmoidal shape, conical shape, concave shape, funnel shape, or parabolic shape. Thereby, sharp edges potentially impairing the insertion are avoided and a gradually extending guiding surface may be provided facilitating the insertion. The shapes are to be understood as a shape substantially extending from the insertion portion defining the corresponding portion of the through-hole, in particular extending from a portion having a substantially continuous diameter, and as seen in a longitudinal sectional view of the respective part. They are typically continuous in the circumferential direction, i.e. do not form gaps, based on their rotational symmetry.

According to some embodiments, the insertion portion and, in particular, the corresponding portion having the increased cross-sectional area of the through-hole may include structures with particular geometries or surface irregularities shaped to form retention surfaces, such as protrusions, slits, threads, grooves or holes on their outer surface. In particular, such external structures may (i) increase the contact between the surface of the insertion portion and the adhesive; and (ii) create anchor points for the adhesive. Such external structures are disclosed for example in patent application WO2022229207 the content of which is incorporated by reference in its entirety.

The provision of the larger cross-sectional area at least at one end of the nerve conduit system hence significantly facilitates the insertion of a respective nerve end of a nerve to be repaired. In particular, the larger cross-sectional area may provide a tolerance of an (unintended) offset of the insertion height of the nerve end with regard to the cross-sectional area of the through-hole of the insertion portion during a repair procedure. Thereby, even if such offset is provided, it is ensured that the nerve end is received within the nerve conduit and (minor) adjustments may be performed without requiring a re-insertion of the nerve end or specific action on the nerve conduit. In addition, the larger cross-sectional area ma serve as an optical guiding surface for a surgeon, thus further facilitating proper insertion of the respective nerve end. Thereby, proper insertion and placement of the nerve end into the nerve conduit system, e.g. into the through-hole, which may be particularly dimensioned to accommodate the nerve ends to be connected, may be achieved more easily and in a time-effective manner and without adversely affecting the respective nerve end.

In some embodiments, to facilitate or direct proper axonal growth from the nerve end having a larger diameter, e.g. being received and accommodated in the first part, to the nerve end having a smaller diameter, e.g. being received in the through-hole of the second part, a guiding surface towards the second diameter and/or the insertion portion of the other (second) part may be provided. Such configuration may also be desirable for the surrounding tissue by providing soft edges or no edges at the interface with the adjacent portions. The conical shape may also be easier to grip during surgery, such that the handling of the first part may be facilitated. Furthermore, the conical shape may improve the structural stability, both, of the first part and the nerve conduit system as a whole in the connected state, i.e. when the connection portions of the first part and of the second part are engaged.

In some embodiments, the first diameter of the through-hole of the insertion portion of the first part may be smaller than the second diameter of the corresponding connection portion. Thereby, the transition portion may define a radially outward expanding through-hole portion. The connection portion may be arranged as a longitudinal extension relative to the insertion portion. The through-hole e.g. of the first part may substantially be formed by the insertion portion, the transition portion and the connection portion. Said through-hole is expanded radially outward in the transition portion. Such configuration may be provided, for example, when the diameter of the nerve end of the first part is smaller than or equal to the nerve end to be accommodated in the second part. By the same token, such configuration may be provided, when the nerve ends to be connected have relatively small diameters, i.e. smaller than the connection portion the first part. Accordingly, nerve ends having substantially the same size or a nerve end of the first part having a larger diameter than the nerve end of the second part may also be connected using such configuration, as long as the diameter of the nerve end received in the first part is smaller than the corresponding connection portion.

If the through-hole of the insertion portion of the first part has a larger first diameter than the second diameter of the corresponding connection portion, the transition portion may define a radially inward reducing through-hole portion. Accordingly, the first part may accommodate a nerve end having a larger diameter than the nerve end being accommodated in the second part.

In some embodiments, the connection portion may have a predefined diameter. Such predefined dimension of the connection portion(s) may be particularly desirable in a kit of parts to accommodate a corresponding connection portion for a variety of second parts having different dimensions, e.g. of the through-hole and/or insertion portion.

It may hence be provided that the connection portion of the first part (and second part) has standardized predefined radial dimension, e.g. in a kit of parts. Thereby, at least the dimensions of the insertions portions and through-hole may be variable. The transition portion of the first part and/or second part may ensure that a gradual transitioning of the terminal end of the nerve end accommodated in the respective insertion portion is directed to the respective connection portion and/or adjacent insertion portion of the respective other part.

In some embodiments, the connection portions are configured to connect the respective through-hole and the overall through-holes in the connected state, i.e. when the connection portions of the first part and of the second part are engaged, towards the exterior of the system. Thereby, axonal growth from one nerve end to the other nerve end through the nerve conduit system may be further facilitated, by preventing axonal escape and allowing nutrient and growth factor concentration. Thus, the risk of axonal growth e.g. around the insertion portion of the first or second part may be reduced or effectively avoided.

At least a portion of the transition portion adjacent to the insertion portion (particularly the entire transition portion) may comprise or be formed by a wall, wherein said wall is arranged at a predefined angle relative to a wall of the insertion portion and wherein the predefined angle is based on a difference and a distance between the first diameter (of the through-hole) of the insertion portion and the second diameter of the corresponding connection portion. In some embodiments, the wall of the transition portion defines a portion of the through-hole. It may also define a bridging portion between the insertion portion and a connection portion arranged along an exterior of the insertion portion, e.g. in a concentric manner.

Accordingly, the length or extension of the transition portion in the longitudinal direction may increase with a corresponding increase in difference between the first and second diameter. To reduce the overall length of the nerve conduit system, the longitudinal extension of the insertion portion of the first part and/or the second part and/or the predefined angle may be accordingly adapted.

In some embodiments, in an arrangement wherein the transition portion surrounds or extends over the respective insertion portion, the predefined angle is between 10 degrees and 60 degrees, between 30 degrees and 60 degrees or between 30 degrees and 50 degrees. In an arrangement, wherein the transition portion is arranged adjacent to the insertion portion, the predefined angle may be between 120 degrees and 240 degrees or between 130 degrees and 230 degrees.

Such angle may define a conical shape of the transition portion and a funnel shape of the first part (and/or the second part) in a longitudinal section as a whole. By the predefined angle, a predefined, gradual slope is provided, wherein the angle between 120 degrees and 240 degrees has been found to be particularly desirable.

In an alternative, the transition portion (of the first part) may be formed by a wall defining a predefined longitudinal extension, wherein an angle between said wall and a wall forming the insertion portion and/or through-hole thereof is based on a difference between the first diameter and the second diameter. The predefined longitudinal extension or axial length may provide that the overall length of the first part may be standardized for a variety of dimensions of the insertion portion and the connection portion. Such standardized length may also define a predefined gap or spacing between the terminal ends of the nerve ends to be connected via the nerve conduit system. For example, the nerve end accommodated in the first part may be inserted up to or about the interface to the transition portion while the nerve end accommodated in the second part may be inserted up to the end face of the respective insertion portion or up to an interface with an optionally present radially expanding end portion of said insertion portion.

Furthermore, the predefined length may facilitate handling by a surgeon upon implantation of the nerve conduit system, e.g. during insertion of the respective nerve end and/or connection of the first part and the second part.

To facilitate the connection and provide the surgeon with a haptic feedback, the connection portion of the first part may define a stop, wherein said stop is configured to abut the connection portion of the second part when the connection portions of the first part and of the second part are engaged, i.e. in the connected state. The stop may hence prevent the connection portion of the second part to be inserted or advanced beyond said stop in a longitudinal direction, i.e. to avoid that said second part is inserted beyond an intended position. In some embodiments, said stop defines a predefined relative arrangement between the first and second stop. For example, the stop may establish that a predefined spacing or gap is provided between the accommodated nerve ends, wherein said spacing or gap is optimized for the end-to-end connection and nerve regeneration. The stop may furthermore provide a predefined connection, e.g. by a corresponding interference fit and/or by ensuring a positive locking or snap fit, wherein one or more protrusions and corresponding recesses or slits of the first and second part engage each other. The stop may be formed e.g. by a wall section of the connection portion and/or a wall section of an adjacent transition portion, if such transition portion is present.

In some embodiments, the stop is configured to at least partially receive the connection portion of the second part and/or comprises a longitudinally extending detent configured to radially secure the connection portion of the second part when the connection portions of the first part and of the second part are engaged, i.e. in the connected state. Accordingly, while an abutment may be provided e.g. by a radially extending raised portion of a wall of the connection portion and/or adjacent transition portion, the stop is formed to also at least partially surround a directly adjacent end section of the connection portion of the second part, e.g. wall section. The stop may e.g. be formed as a semi-circular or parabolic groove or undercut, in some embodiments, comprising a circumferential wall portion that extends in the radial direction along the adjacent end face of the connection portion of the second part.

As described above, the connection portion may be substantially formed as a circumferential wall extending in the longitudinal direction and being configured to engage a corresponding circumferential wall portion of the connection portion of the second part. The stop may hence form a wall section or detent in parallel to the circumferential wall, such that the end face of the connection portion is radially secured in the inward and outward direction and is received within said undercut.

In some embodiments, in addition or alternatively to the above-described stop of the first part, the second part may (also) define a stop. The above description of the stop of the first part applies accordingly to the stop of the second part. It is understood that the stop of the second part is configured to abut the connection portion of the first part when the connection portions of the first part and of the second part are engaged, i.e. in the connected state. The stop may hence prevent the connection portion of the first part to be inserted or advanced beyond said stop in a longitudinal direction, i.e. to avoid that said second part is inserted beyond an intended position. In some embodiments, the first and the second part each comprise a stop, for example in a complementary manner. In some embodiments, the stop of the first part may be located on the inner surface of the (circumferential) wall of the connection portion of the first part, while the stop of the second part may be located on the outer surface of the (circumferential) wall of the connection portion of the second part; or the stop of the first part may be located on the outer surface of the (circumferential) wall of the connection portion of the first part, while the stop of the second part may be located on the inner surface of the (circumferential) wall of the connection portion of the second part ;.

During the insertion of the nerve ends into the respective nerve conduit part and the subsequent securing of said nerve ends with e.g. a medical adhesive, undesirable compression of the respective nerve conduit part and nerve end may occur, which may be detrimental for the nerve end integrity and repair. To facilitate handling, also during the connection of the first part and the second part, at least one connection and/or transition and/or insertion portion may comprise one or more gripping ribs, which protrude radially outward of the outer surface of a wall of said connection portion and which longitudinally extend at or along the outer surface of the connection portion. By the radially outward extension, the respective part may be easier to grip and handle without directly interfering with the integrity of the insertion portion, through-hole and connection portion, and undesirable actions on the respective part may be absorbed or at least be dampened.

The connection portion may comprise two or more gripping ribs. Accordingly, the connection portion may comprise a single gripping rib or more than one gripping rib, e.g. 2, 3, 4, 5, 6, 7, 8, 9 or more. In some embodiments, when two or more gripping ribs are present, two or more gripping ribs may be equally spaced apart to each other along a circumference of the connection portion. For example, the gripping ribs may be arranged as two or more pairs of ribs, wherein the gripping ribs of each pair have the same predefined circumferential spacing and said pairs may comprise a different predefined circumferential spacing between each other.

The predefined arrangement of the gripping ribs may facilitate orientation during orientation of the respective part and may e.g. provide a level of rotational symmetry and/or redundancy. Furthermore, if both parts comprise one or more ribs, the ribs may be provided at predefined positions, e.g. relative to one or more protrusions or slits of the connection portions, such that the ribs may indicate an intended predefined arrangement of the parts relative to each other. In other words, such arrangement of ribs may facilitate correct alignment of the parts.

The ribs may have a variety of shapes that are desirable for the handling and gripping of the respective part. Since the ribs may increase the radial dimensions of the respective part and/or the nerve conduit system as a whole, the ribs may protrude into the surrounding tissue upon implantation. Accordingly, to avoid or reduce potential tissue damage, each gripping rib may comprise at least one rounded end in a longitudinal section of the respective connection portion. Thereby, sharp edges may be avoided. At least a wall of the connection portion of the first part may furthermore comprise a transparent window or opening, e.g. a radial opening, which may be arranged at a predefined longitudinal offset to a free end of the connection portion of the first part. By the window or opening, an insertion length or depth of the connection portion of the second part may be monitored. Thereby, the surgeon may be given a visual feedback regarding the connected state, i.e. when the connection portions of the first part and of the second part are engaged, and may e.g. insert the connection portion of the second part so as to provide a predefined gap for the nerve ends to be connected and/or according to the therapeutic needs. According to special embodiment, the first part and/or second part of the nerve conduit system are made with transparent material.

The window or opening may form an alternative to a haptic feedback, e.g. provided by protrusions and slots of the connection portions or a stop of the first part. However, the window or opening may also provide an additional feedback to confirm correct positioning and coupling of the nerve conduit parts.

In some embodiments, the window or opening is formed at a wall defining the connection portion, e.g. a circumferential wall extending in the longitudinal direction. For improved structural stability, a transparent window may be provided, for which no sealing with e.g. a medical adhesive may be required. By enabling such monitoring within the connection portion and/or towards the through-hole, the surgeon may hence be provided with a direct visual feedback regarding a distance of the nerve end being received and accommodated by the second part relative to the nerve end received and accommodated in the insertion portion of the first part.

In some embodiments, at least one of the parts, in particular its respective lumen or through-hole, may contain a drug, e.g. by coating or integrated in the material, e.g. a wall, of the respective part, which may be released over time and may e.g. facilitate nerve growth.

In some embodiments, the first part and/or the second part, particularly each part, is formed of a biocompatible material, an inert material, a bioimplantable material, and/or a biodegradable material. The material may be chosen so as to provide a predefined structural stability while substantially avoiding or at least reducing the inflammatory response of a patient to be treated. For example, a biocompatible material may be chosen, which is gradually degrading over time after implantation yet which may initially provide sufficient structural support to adequately repair a nerve lesion and ensure that the respective nerve ends are connected properly and with sufficient stability, e.g. during movement of the tissue.

The particular material may furthermore be chosen in order to facilitate or support nerve growth, for example, by comprising or otherwise incorporating or including a corresponding coating, e.g. with a biologically active agent and/or one or more neurotrophic factors. Other examples of such biologically active surface functionalities include, but are not limited to, e.g. anti-inflammatory immunosuppressants, and neuroprotective agents. Biologically active agents may be surface bound and/or be entrapped in a structure defining the elongate body, e.g. the wall described in the above.

In some embodiments, the first part and/or the second part, particularly each part, is formed of a polymer-based material, particularly an elastomer. This has the aspect that a plurality of manufacturing methods may be applied and/or particular characteristics often material may be provided based on e.g. a polymer unit. In particular, the polymer-based material may be a biocompatible material, which furthermore has elastic properties, such that, in the implanted state, the nerve conduit may adapt to tissue movement surrounding the repaired nerve lesion.

In some embodiments, the first part and/or the second part, particularly each part, may be formed of a polymerized and/or crosslinked polymer unit comprising an ester group component and an acid ester group component, the ester group component being a polyol and the acid ester group component being a polyacid.

The material being used for the respective portions may be the same. Thereby, manufacturing may be further facilitated and structural characteristics of the respective parts may be substantially homogeneous along the longitudinal direction of the nerve conduit system. In this matter, if the nerve conduit system is configured accordingly, biodegradation (and/or bioresorption) may also occur in a predefined or expected manner.

In some embodiments, the first part and/or the second part, particularly each part, is integrally formed (i.e. formed as a single piece each). Accordingly, the connection portions do not require particular connections with e.g. a transition portion or insertion portion. Instead, a material bonding may be provided by the material used for each of the portions and corresponding structural integration. By providing each part as a single piece, the robustness of the nerve conduit system may be further improved since separate connections between portions are effectively avoided.

In some embodiments, the first part and/or the second part, particularly each part, is formed by a 3D-printing process. This is particularly desirable when the material of the nerve conduit is polymer-based, wherein a curing of the material may be provided substantially instantaneously, for example using (UV) light. Furthermore, this provides an accuracy level that may not be (easily) achieved by extrusion and/or a dipping process. In particular, the 3-D printing process enables to obtain a particular shape of each nerve conduit part, wherein, for example, the printing process may provide that particular biologically active agents are integrated in the 3-D structure according to a predefined pattern, e.g. within a mesh structure and/or in particular pockets or cavities formed by the 3-D structure. Thereby, orchestration and support of the nerve repair may be further improved and/or biodegradation may be achieved in a more controllable matter.

The above object is furthermore achieved by use of a nerve conduit described in the above for repairing, supporting, and/or guiding neural tissue, in particular for repairing a peripheral nerve lesion and/or for connecting two nerve ends. Furthermore, the nerve conduit may be used in combination with a medical adhesive. According to an embodiment, the medical adhesive is based on polymer unit comprising an ester group component and an acid ester group component, the ester group component being a polyol and the acid ester group component being a polyacid ; according to one embodiment, the medical adhesive is based on Photocurable Elastomers from Poly(glycerol-co-sebacate), based on poly(glycerol-co-sebacate) acrylate (PGSA).

In a further aspect, a kit of parts is provided, comprising a nerve conduit system according to the present disclosure as described herein, wherein the kit comprises two or more first parts being adapted for different nerve end diameters and/or two or more second parts being adapted for different nerve end diameters, wherein the connection portions of said parts are adapted such that each first part of the kit is connectable to each second part of the kit.

In some embodiments, the kit comprises two or more (different) first parts being adapted for different nerve end diameters and at least one second part. In some embodiments, the kit comprises at least one first part and two or more (different) second parts being adapted for different nerve end diameters. In some embodiments, the kit comprises two or more (different) first parts being adapted for different nerve end diameters and two or more (different) second parts being adapted for different nerve end diameters. The two or more (different) first and/or second parts differ usually in the (dimensions of the) insertion portions. In particular, to accommodate nerve ends of different diameters, the two or more (different) first parts may differ in the (inner) diameter of the insertion portion (through hole). Likewise, to accommodate nerve ends of different diameters, the two or more (different) second parts may differ in the (inner) diameter of the insertion portion (through hole). Thereby, the appropriate size fitting the nerve ending best can be selected among the differently sized (insertion portions of the different) first parts and/or second parts.

In some embodiments, the inner diameter for nerve endings, is selected from 1.5 mm to 12 mm. More particularly, it can be from 1.5 mm to 12 mm for nerves of above wrist; from 1.5 mm to 3 mm for nerves of wrist and hand; and 1.5 mm to 2 mm for nerves of hand.

On the other hand, as described above, the connection portions of each part comprised in the kit are designed such that each first part of the kit can be connected to each second part of the kit. To this end, the connection portions are equally dimensioned. In other words, (the dimensions of) the connection portions of the (different) first parts are the same, at least for the portion of the connection portion, which is involved in the connection with the second part. Furthermore, (the dimensions of) the connection portions of the (different) second parts are the same, at least for the portion of the connection portion, which is involved in the connection with the first part. In this context, it is understood that the connection portions of the first and second parts of the kit are configured to engage each other.

By the particular configuration of the differently sized insertion portions, nerve ends of different diameters may be easily connected by selecting the first and/or second part having the appropriate size of the insertion portion, while the connection portions still fit to each other. Accordingly, the appropriate first and second part may even be selected in situ, e.g. during surgery. This avoids ill-fitting conduits, which may result in difficulties during surgery as well as thereafter.

In a further aspect, a kit of parts is provided, comprising a nerve conduit system according to the present disclosure as described herein, wherein the kit further comprises a medical adhesive. According to an embodiment, the medical adhesive is based on pre-polymer unit comprising an ester group component and an acid ester group component, the ester group component being a polyol and the acid ester group component being a polyacid. According to one embodiment, the medical adhesive is based on Photocurable Elastomers from Poly(glycerol-co-sebacate), based on poly(glycerol-co-sebacate) acrylate (PGSA). According to an embodiment, the medical adhesive is a light-curable adhesive, i.e. polymerizes or otherwise cures upon receiving appropriate radiant energy, more particularly in the form of light from a light source. In some embodiments, the light-curable adhesive comprises a pre-polymer and a photoinitiator, said photoinitiator being able to induce polymerization of the said pre-polymer when exposed to light of a specific wavelength, e.g. ultraviolet (UV) radiations.

According to another aspect of the present disclosure, a method for connecting two nerve ends is provided, comprising the steps of:
providing a first and second part of a nerve conduit system as described in the above;
inserting one of the nerve ends to be connected into the insertion portion of the first part and the one of the nerve ends to be connected into the insertion portion of the second part;
optionally, securing said nerve ends to said respective parts; and
connecting the first and second part in an end-to-end manner by connecting the connection portion of the first part to the connection portion of the second part, by accommodating at least a portion of the insertion portion of the second part in the through-hole of the first part.

The securing of the lesioned nerve ends may be performed by applying a medical adhesive outside of the respective insertion portion. The connection of the first and second part is performed by inserting and/or accommodating the connection portion of the second part into or around the connection portion of the first part until the connection portions engage each other, by interference fit and/or positive locking.

### BRIEF DESCRIPTION OF THE DRAWINGS

The present disclosure will be more readily appreciated by reference to the following detailed description when being considered in connection with the accompanying drawings in which:

FIG. 1 shows a schematic depiction of an exemplified nerve conduit system according to the present disclosure in a connected state, i.e. when the connection portions of the first part and of the second part are engaged, of a first part and a second part in a longitudinal section;

FIG. 2 shows a schematic depiction of the second part according to FIG. 1 in a longitudinal section according to FIG. 1;

FIG. 3 shows a schematic depiction of the nerve conduit according to FIG. 2 with an alternative insertion portion;

FIG. 4 shows a schematic depiction of the nerve conduit according to FIG. 2 with an alternative insertion portion;

FIG. 5 shows a schematic depiction of the first part of a nerve conduit system in a longitudinal section according to another embodiment;

FIG. 6 shows a schematic depiction the first part in a longitudinal section according to another embodiment;

FIG. 7 schematically depicts the first part according to FIG. 6 with an alternative connection portion and/or transition portion;

FIG. 8 schematically depicts the first part according to FIG. 6 with an alternative connection portion and/or transition portion;

FIG. 9 schematically depicts the second part according to FIG. 4 with additional ribs according to an alternative embodiment;

FIG. 10 schematically depicts the second part according to FIG. 4 with additional ribs according to another alternative embodiment;

FIG. 11 shows a schematic depiction the first part of a nerve conduit system in a longitudinal section according to another embodiment with alternative rib arrangements;

FIG. 12 shows a schematic depiction the first part of a nerve conduit system in a longitudinal section according to another embodiment with alternative rib arrangements;

FIG. 13A shows a schematic depiction the first and second part of a nerve conduit system according to an embodiment with positive locking in a top view illustrating both parts in a separate manner and in the connected state (dashed lines); and

FIG. 13B shows a schematic depiction the first and second part of a nerve conduit system according to an embodiment with positive locking in a longitudinal section along A-A, as indicated in FIG. 13A.

### DETAILED DESCRIPTION

In the following, the present disclosure will be explained in more detail with reference to the accompanying figures showing exemplified embodiments of the present disclosure. However, the present disclosure shall not to be limited in scope by the specific embodiments described herein. The following embodiments are given to enable those skilled in the art to more clearly understand and to practice the present disclosure. In the Figures, like elements are denoted by identical reference numerals and repeated description thereof may be omitted in order to avoid redundancies.

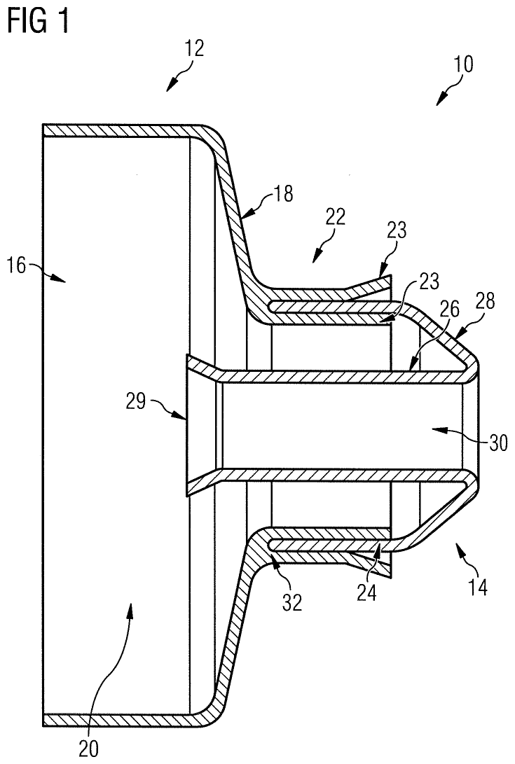

In FIG. 1 a nerve conduit system 10 according to the present disclosure is schematically shown along a longitudinal section. The nerve conduit system 10 comprises a first part 12 and a second part 14, which are depicted here in the connected state, i.e. when the connection portions of the first part and of the second part are engaged. Both the first part 12 and the second part 14 are configured to receive a respective nerve end (not shown), wherein one nerve end constitutes a proximal nerve end and the other constitutes a distal or target nerve end. In the present example, the first part 12 is dimensioned for a nerve end having a larger diameter compared with the nerve end to be received by the second part 14, such that axonal growth may be facilitated from the first part 12 to the second part 14, and vice versa.

Both the first part 12 and the second part are substantially tubular shaped and formed by a wall defining a lumen for receiving and accommodating a respective nerve end. Accordingly, the first part 12 comprises an insertion portion 16 and connection portion 22, which are both defined by the wall. The insertion portion 16 and connection portion 22 are connected via a transition portion 18, forming a continuous and homogeneous structure.

As shown, the insertion portion 16 or wall thereof defines an opening through-hole 20, which is depicted here to extend between the longitudinally opposing ends of the first part 12 and is defined by the insertion portion 16, the transition portion 18, and the connection portion 22. The through-hole 20 is hence be formed as a continuous channel from one end portion of the first part 12 to the opposing end portion.

At the connection portion 22, a two circumferential walls 23 are provided at a radial spacing, which is configured for engaging a corresponding connection portion 24 of the second part 14 in the connected state of the nerve conduit system 10, i.e. when the connection portions 22, 24 of the first part 12 and of the second part 14 are engaged, as shown in the example. The outer wall 23 of the connection portion 22 is connected to the transition portion 18 and defines a longitudinal groove together with the inner wall 23 to receive a corresponding circumferential wall of the connection portion 24 of the second part 14. To facilitate the insertion of the connection portion 24 into said groove and hence facilitate the connection or engagement of the connection portions 22, 24, the wall of the connection portion 22 comprises a radial flare at its free end, thereby providing an enlarged opening of said groove.

At the junction between the transition portion 18 and the connection portion 22 a stop 32 is defined by a wall section of the transition portion 18 and/or the walls 23 of the connection portion 22, which provides an abutment of the connection portion 24 of the second part 14 and ensures that a connection of the first part 12 and the second part 14 may be provided according to a predefined connected state, i.e. when the connection portions 22, 24 of the first part 12 and of the second part 14 are engaged. The hence provided double wall feature formed by the connection portion 22 radially secures the second part 14 to the first part 12 and provides that the first part 12 and second part 14 are at a predefined relative position in the connected state, i.e. when the connection portions 22, 24 of the first part 12 and of the second part 14 are engaged, by the stop 32.

Within the second part 14, a nerve end having a smaller diameter may be received, which is facilitated by the corresponding insertion portion 26, which defines an opening to a through-hole 30 of the second part 14. Also provided is a radially expanding end portion 29 at the longitudinal end of the insertion portion 26 opposing a longitudinal end being configured as an inlet for the respective nerve end. The radially expanding end portion 29 is configured to direct nerve growth from the first part 12, i.e. the insertion portion 16 and/or transition portion 18 into the through-hole 30. In some embodiments, the nerve end to be accommodated within the second part 14, is inserted into the through-hole 30, which is substantially defined by the insertion portion 26, up to the radially expanding end portion 29. By the same token, the nerve end accommodated in the through-hole 20 of the first part 12 is also inserted in such manner, that said nerve end does not extend beyond the respective insertion portion 16, i.e. does not extend into the transition portion 18. Accordingly, the nerve ends are brought into proximity of each other in an end-to-end manner, wherein the transition portion 18 of the first part 12 and the radially expanding end portion 29 of the second part 14 provide a predefined spacing or gap between said nerve ends.

The connection portion 24 of the second part 14 shown in this example extends from the insertion portion 26 in a longitudinal direction along an outer surface of a wall of the respective insertion portion 26 and is configured to seal the through-hole 30 at a distal end of the first part 12. Accordingly, nerve regeneration is contained within the nerve conduit system 10 and is effectively directed from the insertion portion 16 of the first part 12 to the insertion portion 26 of the second part 14. In other words, axonal growth between the first part 12 and the second part 14 outside of the through-hole 30 is avoided to the largest extent.

The arrangement of the connection portion 24 at a radial spacing from the insertion portion 26 of the second part 14 ensures that the connection portion 24 may engage the corresponding connection portion 22 of the first portion 12. At the same time, the difference in diameter between the connection portion 24 and the insertion portion 26 provides that the insertion portion 26 may be received and accommodated by the connection portion 22 or portion of the through-hole 30 defined by said connection portion 22. The insertion portion 26 may hence extend towards or into the transition portion 18 of the first part 12 so as to facilitate the end-to-end connection of the respective nerve ends. Furthermore, this provides that structural stability may be improved for the second part 14 without significantly increasing the overall dimensions of the nerve conduit system 10 as a whole.

The connection portion 24 of the second part 14 is connected to the insertion portion 26 by a corresponding transition portion 28, which extends between the connection portion 24 and an inlet end and/or proximal end of the insertion portion 26. Accordingly, the transition portion 28 bridges a first diameter at the junction with the insertion portion 26 with a second diameter at the junction with the connection portion 24. The connection portion 24 surrounds the insertion portion 26 in a substantially concentric manner.

As shown in FIG. 1, the sealing of the through-hole 30, i.e. the space between the inner surface of the inner wall 23 of the connection portion 22 and the outer surface of the insertion portion 26 primarily sealed at the distal end of the first part 12 is done by the transition portion 28. Such space may furthermore reduce potential undesirable friction and/or provide a bearing for the second part 14, which dampens radial forces, e.g. during surgery or during movement at the implantation site.

By the same token, the transition portion 18 extends between the insertion portion 16 and the connection portion 22 and bridges different diameters of the respective portions 16, 22. In such adjacent arrangement, the transition portion 18 defines a corresponding portion of the through-hole 20. Furthermore, the transition portion 18 comprises a substantially conical shape, providing a gradual guiding surface towards the radially expanding end portion 29 and hence facilitating axonal growth towards the nerve end being accommodated in the insertion portion 26 of the second part 14.

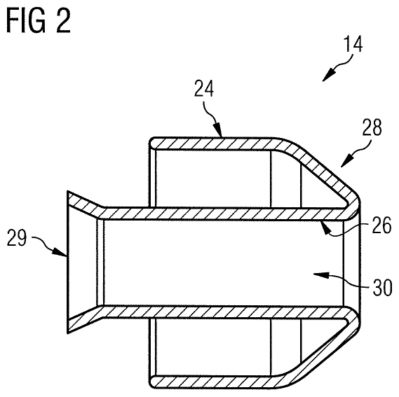

In FIG. 2 the second portion 14 according to FIG. 1 is depicted in an uncoupled or disconnected state, indicating a free end of the connection portion 24 that is to be received and engages the connection portion 22 of the first part 12. From this Figure it is also apparent that the transition portion 28 extending from the insertion portion 26 forms a sealing between the longitudinally extending wall of the connection portion 24 and the longitudinal extending wall of the insertion portion 26 defining the through-hole 30.

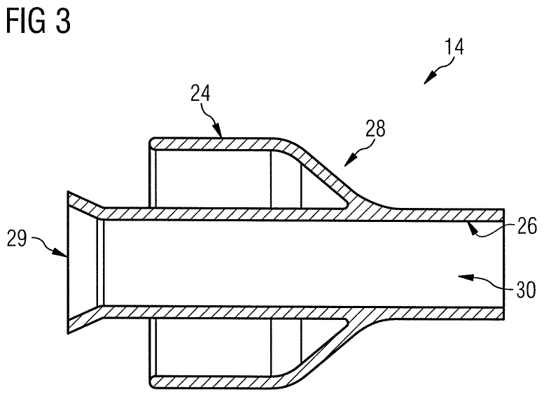

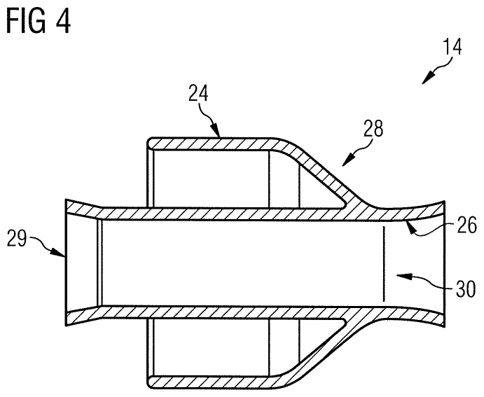

In FIGS. 3 and 4 alternative embodiments of the second part 14 according to FIG. 2 are schematically depicted, wherein the insertion portion 26 has been configured differently. Accordingly, as shown in FIG. 3, the insertion portion 36 may be extended in the longitudinal direction, substantially forming an elongation of the through-hole 30. The connection portion 24 is hence arranged at a predefined longitudinal offset from an inlet end of the insertion portion 26. Such embodiment may be desirable for applications requiring a longer nerve lesion gap to be bridged by the nerve conduit system 10, wherein the connection portion 24 is substantially provided in the same manner so as to reduce the overall (radial) dimensions of the second part 14 and to ensure that the connection of the first part 12 and second part 14 is not impaired by a corresponding extension of the connection portion 24. In other words, a predefined flexibility or resilience may be maintained for the connection portion 24 while the second part 14 is adapted to bridge a longer nerve lesion gap.

The embodiment according to FIG. 4 substantially corresponds to the embodiment according to FIG. 3. However, in this configuration, the insertion portion 26 comprises an opening and cross-sectional area that increases in a direction away from the connection portion 24. The insertion portion 26 is formed as a conical portion, thereby facilitating the insertion of a respective nerve end into the insertion portion 26.

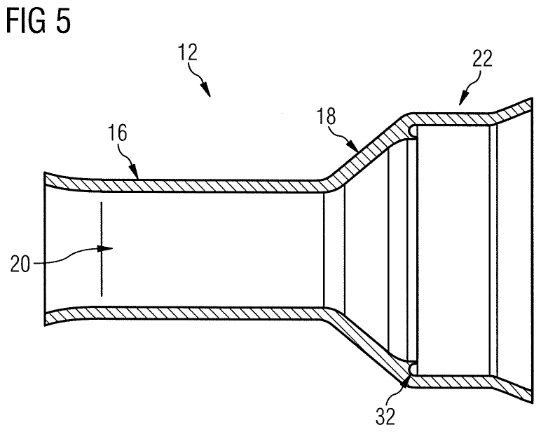

In FIG. 5 a schematic depiction of the first part 12 according to the present disclosure is shown in a longitudinal section according to another embodiment. In comparison with the embodiment according to FIG. 1, an insertion portion 16 is provided, which is accordingly adapted to receive a nerve end having a smaller diameter compared with the embodiment according to FIG. 1. The insertion portion 16 is formed as a substantially cylindrical portion, wherein a radially outward expanding opening is provided at an inlet end or proximal end, which is conically or parabolically shaped. Thereby, insertion of nerve ends with smaller diameters into the insertion portion 16 may be facilitated, as described above e.g. in view of the insertion portion 26 of the second part 14 according to FIG. 4.

In accordance with the smaller dimensioned insertion portion 16 and through-hole 20, the transition portion 18 according to this example extends radially outward to connect the smaller through-hole 20 with a larger dimensioned connection portion 22. The connection portion 22 according to the present example is formed as a single circumferential wall extending in the longitudinal direction of the first part 12 and away from the transition portion 18.

Also depicted in the embodiment according to FIG. 5 is the stop 32, which is defined as a semi-circular wall extension or detent of the connection portion 22 and/or transition portion 18 and forms a recess to receive and radially secure a corresponding connection element or wall section of the connection portion 24 of the second part 14. The stop 32 defines a longitudinal detent, which is dimensioned so as to avoid that an end face of the connection portion 24 of the second part 14 is inadvertently advanced towards the transition portion 18 and is hence inserted beyond the intended position.

The connection portion 22 is configured to receive the connection portion 24 at the inner surface of the (single) circumferential wall defining the connection portion 22 and forms a guiding surface by enabling a slidable engagement with the connection portion 24 of the second part 14.

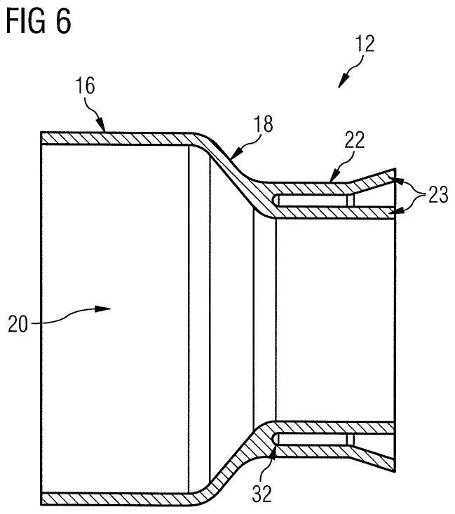

The embodiment according to FIG. 6, which depicts the first part 12, substantially resembles the embodiment depicted in FIG. 1, yet comprises a smaller diameter of the portion of the through-hole 20 defined by the insertion portion 16. In this example, the longitudinal extension of the transition portion 18 has been predefined, such that the angle of the transition portion 20 is less steep compared with the angle depicted in FIG. 1. This is because the difference in diameter between the connection portion 22 and the insertion portion 16 is less and the height or radial extension of the connecting transition portion 18 is hence reduced in comparison with the embodiment depicted in FIG. 1.

Furthermore, similar to the embodiment of the first part 12 according to FIG. 1, the first part 12 according to FIG. 6 is also configured to receive a nerve end having a large diameter compared with the nerve end to be accommodated in the second part 14. The optional enlarged opening at the inlet of the insertion portion 16, as shown e.g. in FIG. 5, is not depicted in this embodiment.

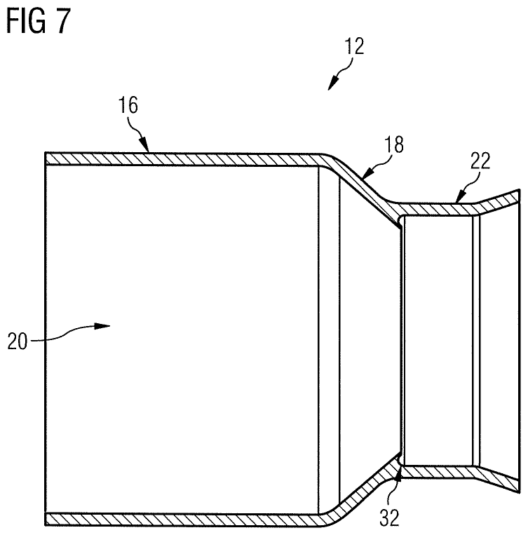

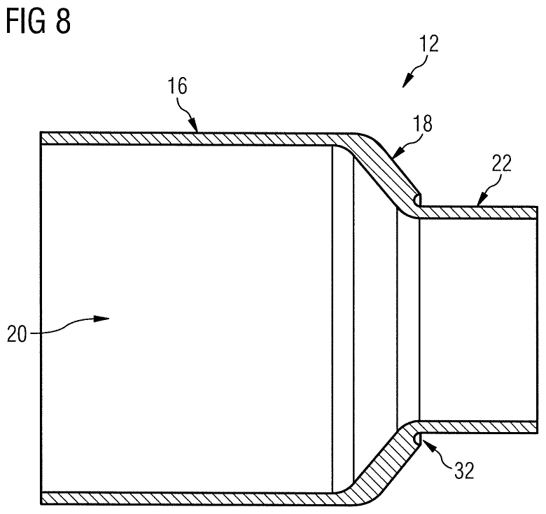

FIGS. 7 and 8 schematically depict the first part 12 according to FIG. 6 with an alternative transition portion 18 and connection portion 22.

Accordingly, as shown in FIG. 7, the transition portion 18 and connection portion 22 may be configured as shown in FIG. 5. However, in this embodiment, the insertion portion 16 and transition portion 18 are configured to accommodate a nerve end having a large diameter relative to the diameter of the nerve end to be accommodated in the second part 14. Hence, the insertion portion 16 and transition portion 18 are formed substantially as depicted in the embodiment according to FIG. 6. Thereby, although the connection portion 22 is similarly configured to accommodate and/or engage the connection portion 24 at the inner surface of the wall defining the connection portion 22, the portion of the through-hole 20 defined by the insertion portion 16 is less pronounced compared with the embodiment according to FIG. 5.

An alternative configuration, wherein the connection portion 24 is received and accommodated at the outer surface of the wall defining the connection portion 22 is depicted in FIG. 8. Compared with the embodiment according to FIG. 7, the free end surface of said wall is not radially expanded, but instead forms a linear extension in parallel with the longitudinal axis of the first part 12 so as to facilitate the coupling at the outer surface of the connection portion 22. According to this embodiment, the transition portion 18 is provided with an increased wall thickness, which may be desirable to improve structural stability and/or to improve the functionality of the stop 32 to impair longitudinal displacement of the connection portion 24 of the second part 14 beyond the transition portion 18 towards the insertion portion 16.

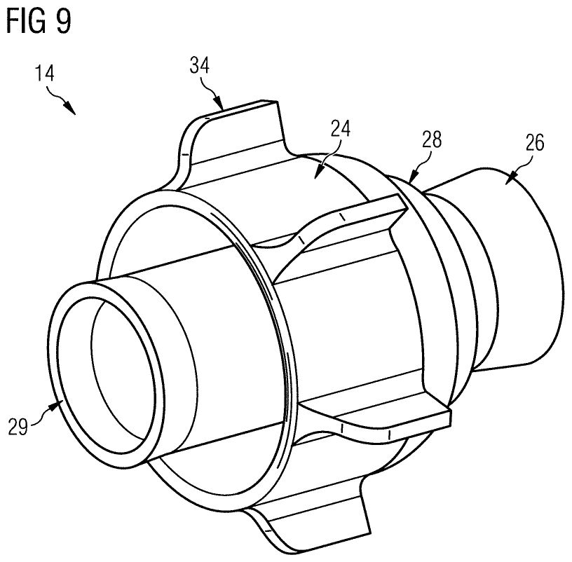

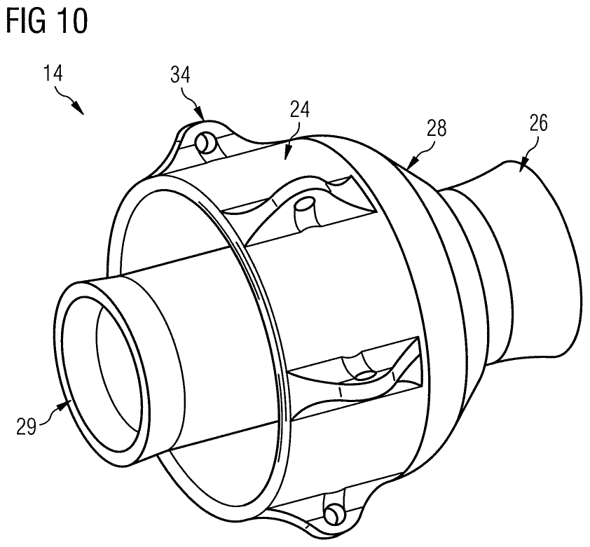

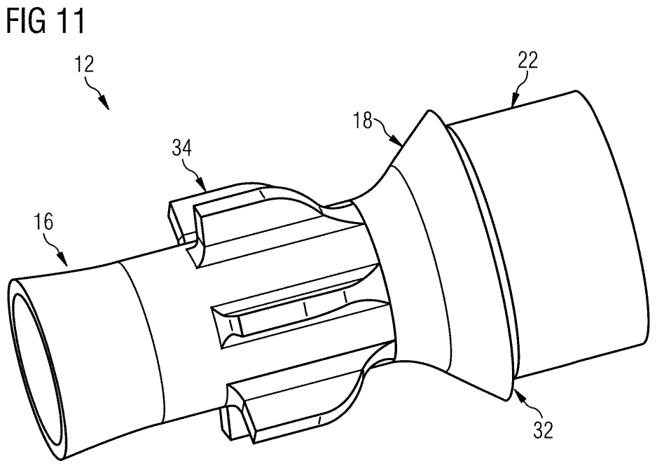

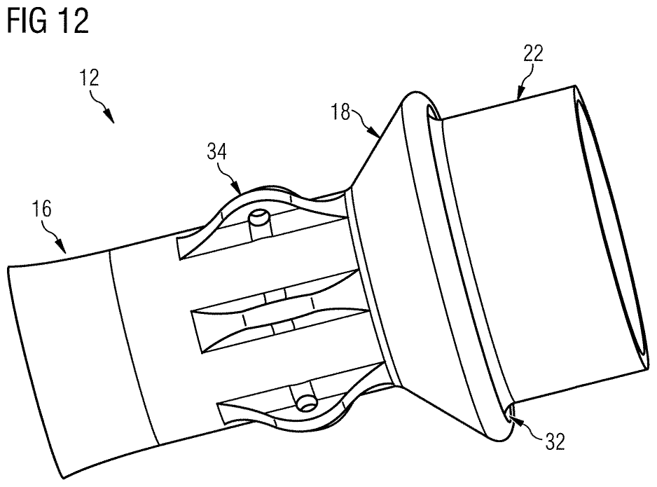

FIGS. 9 and 10 schematically depict the second part 14 according to FIG. 4 with additional ribs 34 according to alternative embodiments. As shown in FIGS. 9 and 10 a plurality of longitudinally extending ribs 34, e.g. 4 to 10 or, as shown, 6 or 8, may be provided at the outer surface of the connection portion 24 of the second part 14, wherein the ribs 34 are equally spaced apart along the circumference of a wall defining the connection portion 24. As shown in FIG. 9, the ribs 34 are rounded at one longitudinal end whereas the ribs 34 are rounded at both longitudinal ends in the embodiment according to FIG. 10. The double rounded surface may be desirable for the surrounding tissue at the target implantation site and may provide a reduced overall dimensioning. However, a single rounded end may also be chosen to improve the potential gripping force and handling surface. FIGS. 11 and 12 show a schematic depiction the first part 12 according to the present disclosure in a longitudinal section according to another embodiment with alternative arrangements of ribs 34 provided at the outer surface of the connection portion 22. The configuration of the first part 12 substantially corresponds to a combination of the embodiments depicted in FIGS. 5 and 8, wherein the insertion portion 16, through-hole 20 and transition portion 18 are dimensioned to accommodate a smaller nerve end diameter while a single circumferential wall also defines the connection portion 22, but wherein the connection portion 22 is adapted to accommodate a corresponding connection portion 24 of the second part 14 at the outer surface of said wall.

As for FIGS. 9 and 10, the first part 12 may also comprise a plurality of spaced-apart ribs 34, e.g. 4 to 10, which are rounded at one or both of the longitudinal ends in accordance with the structural and anatomical needs of the patient at the implantation site.

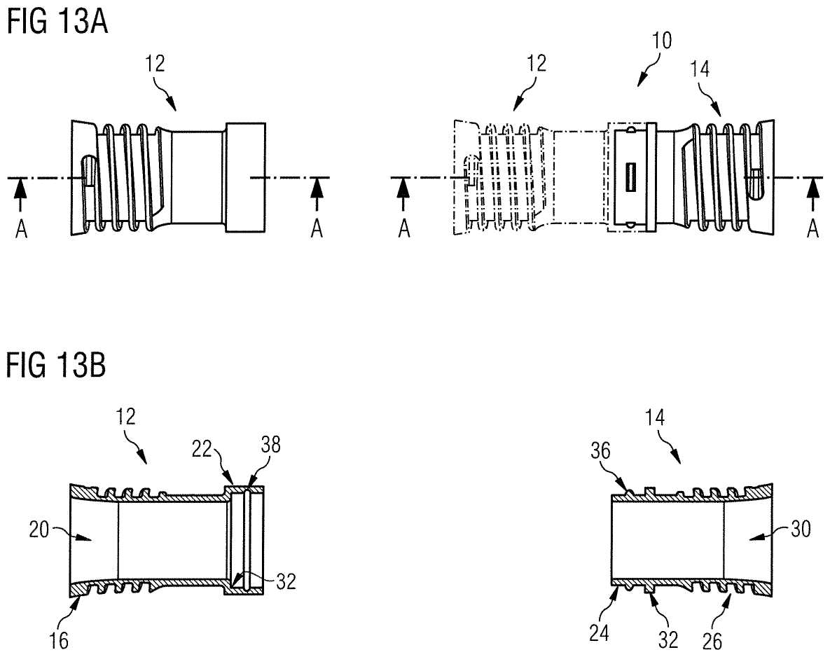

FIGS. 13A and 13B show a schematic depiction the first part 12 and the second part 14 of a nerve conduit system 10 illustrating both parts in a separate manner and in the connected state (dashed lines), wherein the parts 12, 14 are designed such that they may also be secured or fixed to each other by positive locking. Such positive locking may reduce the need for a medical adhesive and/or may facilitate the coupling of the parts 12, 14 and connection of the nerve ends to each other, potentially reducing the occurrence of a loosened engagement.

Accordingly, the connection portion 24 of the second part 14 comprises several radial protrusions 36, while the connection portion 22 of the first part 12 comprises a (corresponding) slit or groove 38 configured to receive said protrusions 36. Thereby, the second part 14 can be at least longitudinally secured to the first part 12 when the connection portions 22, 24 of the first part 12 and of the second part 14 are engaged, i.e. in the connected state. In particular, the at least one radial protrusion 36 and the at least one slit or groove 38 are configured to be in snap fit engagement in the connected state, i.e. when the connection portions of the first part and of the second part are engaged.

The slit or groove 38 of the connection portion 22 of the first part 12 is positioned at an inner (circumferential) wall of said connection portion 22. In addition, the protrusion 36 of the connection portion 24 of the second part 14 is positioned at an outer (circumferential) wall of said connection portion 24. Accordingly, the circumferential wall of the connection portion 24 of the second part 14 may hence be received within or accommodated by the circumferential wall of the connection portion 22 of the first part 12, i.e. being inserted inside a single circumferential wall or into a groove 38 defined by the connection portion 22 of the first part 12. In the connected state, i.e. when the connection portions 22, 24 of the first part 12 and of the second part 14 are engaged, the interfaces at the outer surface of the connection portion of the second part may hence engage the corresponding interfaces at the inner surface of the connection portion of the first part.

In addition, in the embodiment depicted in FIG. 13B, both the first part 12 and the second part 14 each comprise a stop 32 in a complementary manner. Thereby, the stop 32 of the first part 12 may be located on the inner surface of the (circumferential) wall of the connection portion 22 of the first part 12, while the stop 32 of the second part 14 may be located on the outer surface of the (circumferential) wall of the connection portion 24 of the second part 14.

It will be obvious for a person skilled in the art that these embodiments and items only depict examples of a plurality of possibilities. Hence, the embodiments shown here should not be understood to form a limitation of these features and configurations. Any possible combination and configuration of the described features can be chosen according to the scope of the present disclosure.

### LIST OF REFERENCE NUMERALS

10 Nerve conduit system
12 First part
14 Second part
16 Insertion portion
18 Transition portion
20 Through-hole
22 Connection portion
23 Wall
24 Connection portion
26 Insertion portion
28 Transition portion
29 Radially expanding end portion
30 Through-hole
32 Stop
34 Rib
36 Radial protrusion
38 Slit or groove

1. A nerve conduit system (10) for connecting two nerve ends in an end-to-end manner, comprising a first part (12) and a second part (14), each part (12, 14) comprising an insertion portion (16, 26) each being adapted to receive one of the two nerve ends, and a connection portion (22, 24),
wherein each part (12, 14) comprises a through-hole (20, 30) being defined at least by the respective insertion portion (16, 26) and extending between longitudinally opposing ends of the respective part (12, 14) and
wherein the connection portions (22, 24) are configured to engage each other and are arranged such that the through-holes (20, 30) are in fluid communication with each other when the connection portions (22,24) of the first (12) and second part (14) are engaged and optionally wherein, when the first (12) and second part (14) are engaged, the connection portions (22,24) preferably do not act as a pressing member applying a force in the radial direction of the nerve end.

2. The nerve conduit system (10) according to claim 1, wherein at least one part (12, 14), preferably the first part (12) and the second part (14), further comprises a transition portion (18, 28) extending between the respective insertion portion (16, 26) and the respective connection portion (22, 24).

3. The nerve conduit system (10) according to claim 2, wherein the transition portion (18, 28) has an essentially tapered shape.

4. The nerve conduit system (10) according to any one of the preceding claims, wherein the insertion portion (16, 26) and/or the connection portion (22, 24) of at least one part (12, 14), preferably of each part (12, 14), has an essentially cylindrical shape.

5. The nerve conduit system (10) according to any one of the preceding claims, wherein the connection portion (22, 24) of at least one part (12, 14), preferably of the second part (14), at least partially surrounds the insertion portion (16, 26) of said part (12, 14).

6. The nerve conduit system (10) according to claim 5, wherein the insertion portion (16, 26) and the connection portion (22, 24) of at least one part (12, 14), preferably of the second part (14), are arranged in a concentric manner.

7. The nerve conduit system (10) according to any one of the preceding claims, wherein the insertion portion (16, 26) and the connection portion (22, 24) of at least one part (12, 14), preferably of the first part (12), are arranged adjacent to each other in longitudinal direction.

8. The nerve conduit system (10) according to claim 7, wherein a transition portion (18, 28) is arranged between the insertion portion (16, 26) and the connection portion (22, 24).

9. The nerve conduit system (10) according to any of the preceding claims, wherein the connection portions (22, 24) are configured such that the insertion portions (16, 26) are arranged at longitudinally opposing ends of the nerve conduit system (10) when the connection portions (22,24) of the first (12) and second part (14) are engaged.

10. The nerve conduit system (10) according to any of the preceding claims, wherein the through-holes (20, 30) of the first and second part (12, 14) define a single longitudinal axis of the nerve conduit system (10) when the connection portions (22,24) of the first (12) and second part (14) are engaged.

11. The nerve conduit system (10) according to any of the preceding claims, wherein the connection portions (22, 24) are configured such that the through-hole (30) of the second part (14) is at least partially accommodated within the through-hole (20) of the first part (12) when the connection portions (22,24) are engaged.

12. The nerve conduit system (10) according to any of the preceding claims, wherein a transition portion (18) of the first part (12) is configured to at least partially receive and accommodate a radially expanding end portion (29) of the insertion portion (26) of the second part (14) when the connection portions (22,24) of the first (12) and second part (14) are engaged.

13. The nerve conduit system (10) according to any of the preceding claims, wherein the insertion portion (26) and/or a radially expanding end portion (29) of the insertion portion (26) of the second part (14) extends beyond the respective connection portion (24) in the longitudinal direction.

14. The nerve conduit system (10) according to any of the preceding claims, wherein the connection portion (24) of the second part (14) extends from a longitudinal end portion of the respective insertion portion (26) being configured to receive and/or insert the respective nerve end or with a longitudinal offset to a longitudinal end portion of the respective insertion portion being configured to receive and/or insert the respective nerve end.

15. The nerve conduit system (10) according to any of the preceding claims, wherein the connection portion (22) of the first part (12) is arranged adjacent to a respective transition portion (18) or a longitudinal end portion of the respective insertion portion (16) opposing a longitudinal end portion of the respective insertion portion (16) being configured to receive and/or insert the respective nerve end.

16. The nerve conduit system (10) according to any of the preceding claims, wherein at least a portion of the through-hole (20, 30), which is defined by the insertion portion (16, 26) of the first and/or second part (12, 14), is defined by a tubular shaped wall defining an essentially constant inner diameter.

17. The nerve conduit system (10) according to any of the preceding claims, wherein the connection portion (22) of the first part (12) is formed by a single circumferential wall (23) and wherein the single wall (23) accommodates the connection portion (24) of the second part (14) at the inner surface or outer surface of said wall (23).

18. The nerve conduit system (10) according to any of claims 1 to 16, wherein the connection portion (22) of the first part (12) is formed by a circumferential inner and outer wall (23) defining a longitudinally extending groove between said walls (23), wherein the groove is configured to accommodate the connection portion (24) of the second part (14) when the connection portions (22,24) of the first (12) and second part (14) are engaged.

19. The nerve conduit system (10) according to any of the preceding claims, wherein each connection portion (22, 24) comprises at least one circumferential wall (23) and the circumferential walls (23) are configured to engage and radially bias each other and/or are dimensioned and configured to engage each other by interference fit when the connection portions (22,24) of the first (12) and second part (14) are engaged.

20. The nerve conduit system (10) according to claim 19, wherein the circumferential walls (23) are dimensioned such that the wall (23) of the connection portion (24) of the second part (14) is slidably accommodated by at least one circumferential wall (23) of the connection portion (22) of the first part (12) when the connection portions (22,24) of the first (12) and second part (14) are engaged.

21. The nerve conduit system (10) according to any of the preceding claims, wherein one of the connection portions (22, 24) comprises at least one radially extending protrusion (36) and the other connection portion (22, 24) comprises at least one slit or groove (38) configured to receive said protrusion (36) and, preferably, to at least longitudinally secure the second part (14) to the first part (12) when the connection portions (22,24) of the first (12) and second part (14) are engaged.

22. The nerve conduit system according to claim 21, wherein
the at least one protrusion (36) or the at least one slit or groove (38) of the connection portion (22) of the first part (12) is positioned at an inner surface of a circumferential wall (23) of said connection portion (22); and/or
the at least one protrusion (36) or the at least one slit or groove (38) of the connection portion (24) of the second part (14) is positioned at an outer surface of a circumferential wall (23) of said connection portion (24).

23. The nerve conduit system (10) according to claim 21 or 22, wherein the at least one radially extending protrusion (36) and the at least one slit or groove (38) are configured to be in snap fit engagement when the connection portions (22,24) of the first (12) and second part (14) are engaged.

24. The nerve conduit system (10) according to any one of claims 21 to 23, wherein the at least one slit or groove (38) comprises a larger circumferential extension than the at least one protrusion (36).

25. The nerve conduit system (10) according to any of claims 21 to 24, comprising a plurality of protrusions (36) and a plurality of slits or grooves (38), preferably being of equal number, wherein the protrusions (36) and slits or grooves (38) are preferably equally spaced apart in a circumferential direction.

26. The nerve conduit system (10) according to any of the preceding claims, wherein the cross-sectional area of the through-hole (20, 30) of the insertion portion (16, 26) of the first part (12) and/or the second part (14) is gradually increased at a longitudinal end portion for receiving the nerve end and in a direction facing away from the respective connection portion (22, 24).

27. The nerve conduit system (10) according to any of the preceding claims, wherein a transition portion (18, 28) of the first part (12) and/or the second part (14) is configured to steplessly connect the respective insertion portion (16, 26) having a first diameter with the respective connection portion (22, 24) having a second diameter different from the first diameter.

28. The nerve conduit system (10) according to claim 27, wherein at least a portion of the transition portion (18, 28) adjacent to the insertion portion (16, 26) comprises a conical shape or tapered shape in a longitudinal section of the first part.

29. The nerve conduit system (10) according to claim 27 or 28, wherein at least a portion of the transition portion (18, 28) adjacent to the insertion portion (16, 26) comprises a wall, wherein said wall is arranged at a predefined angle relative to a wall of the insertion portion (16, 26), wherein the predefined angle is based on a difference and a distance between the first diameter and the second diameter.

30. The nerve conduit system (10) according to claim 29, wherein the predefined angle is between 30 degrees and 60 degrees, preferably between 40 degrees and 50 degrees, in an arrangement, wherein the transition portion (18, 28) surrounds and/or extends over the respective insertion portion (16, 26) or wherein the predefined angle is between 120 degrees and 240 degrees, preferably between 130 degrees and 230 degrees, in an arrangement, wherein the transition portion (18, 28) is adjacent to the insertion portion (16, 26).

31. The nerve conduit system (10) according to any of the preceding claims, wherein the connection portion (22) of the first part (12) defines a stop (32), said stop (32) being configured to abut the connection portion (24) of the second part (14) when the connection portions (22,24) of the first (12) and second part (14) are engaged.

32. The nerve conduit system (10) according to claim 31, wherein the stop (32) is configured to at least partially receive the connection portion (24) of the second part (14) and/or comprises a longitudinally extending detent configured to radially secure the connection portion (24) of the second part (14) when the connection portions (22,24) of the first (12) and second part (14) are engaged.

33. The nerve conduit system (10) according to any of the preceding claims, wherein at least one part (12, 14) comprises one or more gripping ribs (34), which protrude radially outward of the outer surface of a wall of said connection portion (22, 24) and which preferably longitudinally extend at or along the outer surface of said part (12, 14).

34. The nerve conduit system (10) according to claim 33, wherein the part (12, 14) comprises two or more gripping ribs (34), wherein two or more gripping ribs (34) are preferably equally spaced apart to each other along a circumference of the part (12, 14).

35. The nerve conduit system (10) according to claim 33 or 34, wherein the one or more gripping ribs (34) comprise at least one rounded longitudinal end in a longitudinal section of the respective connection portion (22, 24).

36. The nerve conduit system (10) according to any of the preceding claims, wherein a wall (23) of the connection portion (22) of the first part (12) comprises a transparent window or opening arranged at a predefined longitudinal offset to a free end of the connection portion (22) of the first part (12).

37. A kit of parts, comprising a nerve conduit system (10) according to any of the preceding claims, wherein the kit comprises two or more first parts (12) being adapted for different nerve end diameters and/or two or more second parts (14) being adapted for different nerve end diameters, wherein the connection portions (22, 24) of said parts are adapted such that each first part (12) of the kit is connectable to each second part (14) of the kit.
